SEQUENCE LISTING (35 sequences)

SEQ ID NO: 1 - DNA, 783 bp, Artificial Sequence
  The sequence is synthetized.
atggagtttgggctgagctggctttttcttgtggctattttaaaaggtgtccagtgctctagacaggttcagctggtgcagtctggcagcgagctgaagaaacctggcgcctctgtgaaggtgtcctgcaaggctagcggctacacattcaccgactacagcatgcactgggtccgaaaggcccctggacagggacttgaatggatgggctggatcaacaccgccacaggcgagcctacctacgccgatggcttcacaggcagattcgtgatcagcctggacaccagcgccagaaccgtgtacctgcagatcagctctctgaaggccgaggataccgccgtgtacttctgcttcagctactacgactactggggccagggcaccctggtcacagtttcttctggtggtggtggttctggtggtggtggttctggcggcggcggctccggtggtggtggatccgacatcgtgctgacacagagccctctgagcctgcctgttacacctggcgagcctgccagcatcagctgtagagccagccagaccatctacaagaacctgcactggtatctgcagaagcccggccagtctcctcagctgctgattaagtacggcagcgacagcatctccggcgtgccagatagattcagcggctctggctctggcaccgactacaccctgaagatcagcagagtggaagccgaggacgtgggcgtgtactactgtctgcaaggctacagcaccccttggacattcggccagggcaccaagctggaaatcaag

SEQ ID NO: 2 - protein, 261 aa, Artificial Sequence
  The sequence is synthetized.
MEFGLSWLFLVAILKGVQCSRQVQLVQSGSELKKPGASVKVSCKASGYTFTDYSMHWVRKAPGQGLEWMGWINTATGEPTYADGFTGRFVISLDTSARTVYLQISSLKAEDTAVYFCFSYYDYWGQGTLVTVSSGGGGSGGGGSGGGGSGGGGSDIVLTQSPLSLPVTPGEPASISCRASQTIYKNLHWYLQKPGQSPQLLIKYGSDSISGVPDRFSGSGSGTDYTLKISRVEAEDVGVYYCLQGYSTPWTFGQGTKLEIK

SEQ ID NO: 3 - DNA, 339 bp, Artificial Sequence
  The sequence is synthetized.
caggttcagctggtgcagtctggcagcgagctgaagaaacctggcgcctctgtgaaggtgtcctgcaaggctagcggctacacattcaccgactacagcatgcactgggtccgaaaggcccctggacagggacttgaatggatgggctggatcaacaccgccacaggcgagcctacctacgccgatggcttcacaggcagattcgtgatcagcctggacaccagcgccagaaccgtgtacctgcagatcagctctctgaaggccgaggataccgccgtgtacttctgcttcagctactacgactactggggccagggcaccctggtcacagtttcttct

SEQ ID NO: 4 - protein, 113 aa, Artificial Sequence
  The sequence is synthetized.
QVQLVQSGSELKKPGASVKVSCKASGYTFTDYSMHWVRKAPGQGLEWMGWINTATGEPTYADGFTGRFVISLDTSARTVYLQISSLKAEDTAVYFCFSYYDYWGQGTLVTVSS

SEQ ID NO: 5 - protein, 8 aa, Artificial Sequence
  The sequence is synthetized.
GYTFTDYS

SEQ ID NO: 6 - protein, 8 aa, Artificial Sequence
  The sequence is synthetized.
INTATGEP

SEQ ID NO: 7 - protein, 6 aa, Artificial Sequence
  The sequence is synthetized.
FSYYDY

SEQ ID NO: 8 - DNA, 321 bp, Artificial Sequence
  The sequence is synthetized.
gacatcgtgctgacacagagccctctgagcctgcctgttacacctggcgagcctgccagcatcagctgtagagccagccagaccatctacaagaacctgcactggtatctgcagaagcccggccagtctcctcagctgctgattaagtacggcagcgacagcatctccggcgtgccagatagattcagcggctctggctctggcaccgactacaccctgaagatcagcagagtggaagccgaggacgtgggcgtgtactactgtctgcaaggctacagcaccccttggacattcggccagggcaccaagctggaaatcaag

SEQ ID NO: 9 - protein, 107 aa, Artificial Sequence
  The sequence is synthetized.
DIVLTQSPLSLPVTPGEPASISCRASQTIYKNLHWYLQKPGQSPQLLIKYGSDSISGVPDRFSGSGSGTDYTLKISRVEAEDVGVYYCLQGYSTPWTFGQGTKLEIK

SEQ ID NO: 10 - protein, 11 aa, Artificial Sequence
  The sequence is synthetized.
RASQTIYKNLH

SEQ ID NO: 11 - protein, 7 aa, Artificial Sequence
  The sequence is synthetized.
YGSDSIS

SEQ ID NO: 12 - protein, 9 aa, Artificial Sequence
  The sequence is synthetized.
LQGYSTPWT

SEQ ID NO: 13 - DNA, 783 bp, Artificial Sequence
  The sequence is synthetized.
atggagtttgggctgagctggctttttcttgtggctattttaaaaggtgtccagtgctctagagacatcgtgctgacacagagccctctgagcctgcctgttacacctggcgagcctgccagcatcagctgtagagccagccagaccatctacaagaacctgcactggtatctgcagaagcccggccagtctcctcagctgctgattaagtacggcagcgacagcatctccggcgtgccagatagattcagcggctctggctctggcaccgactacaccctgaagatcagcagagtggaagccgaggacgtgggcgtgtactactgtctgcaaggctacagcaccccttggacattcggccagggcaccaagctggaaatcaagggtggtggtggttctggtggtggtggttctggcggcggcggctccggtggtggtggatcccaggttcagctggtgcagtctggcagcgagctgaagaaacctggcgcctctgtgaaggtgtcctgcaaggctagcggctacacattcaccgactacagcatgcactgggtccgaaaggcccctggacagggacttgaatggatgggctggatcaacaccgccacaggcgagcctacctacgccgatggcttcacaggcagattcgtgatcagcctggacaccagcgccagaaccgtgtacctgcagatcagctctctgaaggccgaggataccgccgtgtacttctgcttcagctactacgactactggggccagggcaccctggtcacagtttcttct

SEQ ID NO: 14 - protein, 261 aa, Artificial Sequence
  The sequence is synthetized.
MEFGLSWLFLVAILKGVQCSRDIVLTQSPLSLPVTPGEPASISCRASQTIYKNLHWYLQKPGQSPQLLIKYGSDSISGVPDRFSGSGSGTDYTLKISRVEAEDVGVYYCLQGYSTPWTFGQGTKLEIKGGGGSGGGGSGGGGSGGGGSQVQLVQSGSELKKPGASVKVSCKASGYTFTDYSMHWVRKAPGQGLEWMGWINTATGEPTYADGFTGRFVISLDTSARTVYLQISSLKAEDTAVYFCFSYYDYWGQGTLVTVSS

SEQ ID NO: 15 - protein, 20 aa, Artificial Sequence
  The sequence is synthetized.
GGGGSGGGGSGGGGSGGGGS

SEQ ID NO: 16 - protein, 25 aa, Artificial Sequence
  The sequence is synthetized.
QVQLVQSGSELKKPGASVKVSCKAS

SEQ ID NO: 17 - protein, 17 aa, Artificial Sequence
  The sequence is synthetized.
MHWVRKAPGQGLEWMGW

SEQ ID NO: 18 - protein, 38 aa, Artificial Sequence
  The sequence is synthetized.
TYADGFTGRFVISLDTSARTVYLQISSLKAEDTAVYFC

SEQ ID NO: 19 - protein, 11 aa, Artificial Sequence
  The sequence is synthetized.
WGQGTLVTVSS

SEQ ID NO: 20 - protein, 23 aa, Artificial Sequence
  The sequence is synthetized.
DIVLTQSPLSLPVTPGEPASISC

SEQ ID NO: 21 - protein, 15 aa, Artificial Sequence
  The sequence is synthetized.
WYLQKPGQSPQLLIK

SEQ ID NO: 22 - protein, 32 aa, Artificial Sequence
  The sequence is synthetized.
GVPDRFSGSGSGTDYTLKISRVEAEDVGVYYC

SEQ ID NO: 23 - protein, 10 aa, Artificial Sequence
  The sequence is synthetized.
FGQGTKLEIK

SEQ ID NO: 24 - DNA, 135 bp, Homo sapiens
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 25 - protein, 45 aa, Homo sapiens
TTTPAPRPPTPAPTIASQPLSLRPEACRPAAGGAVHTRGLDFACD

SEQ ID NO: 26 - DNA, 72 bp, Homo sapiens
atctacatctgggcgcccttggccgggacttgtggggtccttctcctgtcactggttatcaccctttactgc

SEQ ID NO: 27 - protein, 24 aa, Homo sapiens
IYIWAPLAGTCGVLLLSLVITLYC

SEQ ID NO: 28 - DNA, 123 bp, Homo sapiens
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 29 - protein, 41 aa, Homo sapiens
RSKRSRLLHSDYMNMTPRRPGPTRKHYQPYAPPRDFAAYRS

SEQ ID NO: 30 - DNA, 126 bp, Homo sapiens
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 31 - protein, 42 aa, Homo sapiens
KRGRKKLLYIFKQPFMRPVQTTQEEDGCSCRFPEEEEGGCEL

SEQ ID NO: 32 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 33 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - DNA, 1479 bp, Artificial Sequence
  The sequence is synthetized.
atggagtttgggctgagctggctttttcttgtggctattttaaaaggtgtccagtgctctagagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagctgaaacgtggtggtggtggttctggtggtggtggttctggcggcggcggctccggtggtggtggatccgaggtgcagctgcagcagtctggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaagggacctcagtcaccgtctcctcaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 35 - protein, 493 aa, Artificial Sequence [sequence omitted]

(19) United States
(12) Patent Application Publication
(10) Pub. No.: US 2023/0357355 A1
(43) Pub. Date: Nov. 9, 2023

(54) CSPG4-TARGETING HUMANIZED CHIMERIC ANTIGEN RECEPTOR, IMMUNE EFFECTOR CELL EXPRESSING CHIMERIC ANTIGEN RECEPTOR, AND APPLICATIONS THEREOF

(71) Applicant: BOYUAN RUNSHENG PHARMA (HANGZHOU) CO., LTD., Hangzhou (CN)

(72) Inventors: Chuang SUN, Hangzhou (CN); Xin-Hua FENG, Hangzhou (CN); Bin ZHAO, Hangzhou (CN)

(73) Assignee: BOYUAN RUNSHENG PHARMA (HANGZHOU) CO., LTD., Hangzhou (CN)

(21) Appl. No.: 18/028,506

(22) PCT Filed: Sep. 23, 2021
(86) PCT No.: PCT/CN2021/120016

§ 371 Date: Mar. 24, 2023

(30) Foreign Application Priority Data
Sep. 24, 2020 (CN) 202011013925.1

(51) Int. Cl. C07K 14/725; C07K 16/28; C12N 15/86; A61P 35/00

(52) U.S. Cl. CPC C07K 14/7051; C07K 16/28; C12N 15/86; A61P 35/00; C07K 2319/03; C07K 2319/02; C07K 2317/565; C07K 2317/24; C07K 2317/567

(57) ABSTRACT

Related are a CSPG4-targeting humanized chimeric antigen receptor and applications thereof, comprising a humanized anti-CSPG4 binding domain, a hinge region, a transmembrane domain, and a signal transduction domain. The anti-CSPG4 binding domain comprises an anti-CSPG4 antibody or antigen binding part. Related are an immune effector cell expressing the CSPG4-targeting humanized chimeric antigen receptor and applications of the cell.

### CROSS REFERENCE TO THE RELATED APPLICATIONS

This application is the national phase entry of International Application No. PCT/CN2021/120016, filed on Sep. 23, 2021, which is based upon and claims priority to Chinese Patent Application No. 202011013925.1, filed on Sep. 24, 2020, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBZD010_Sequence_Listing.txt, created on Mar. 22, 2023, and is 25,460 bytes in size.

### TECHNICAL FIELD

The present disclosure relates to the field of biomedicine, and particularly to a CSPG4-targeting humanized chimeric antigen receptor, immune effector cells expressing the CSPG4-targeting humanized chimeric antigen receptor, and applications thereof.

### BACKGROUND ART

Cancer is currently one of the diseases with the highest mortality in the world, and in recent years, research on cancer therapy has also shifted from traditional surgery, radiotherapy, chemotherapy to targeted therapy, immunotherapy and other precise therapies. The key to precise therapy is the selection of effective and safe targets.

Chondroitin sulfate proteoglycan 4 (CSPG4), also known as high molecular weight-melanoma-associated antigen (HMW-MAA) or melanoma-associated chondroitin sulfate proteoglycan (MCSP), a member of the chondroitin sulfate proteoglycan family, was first found to be highly expressed in melanoma. In later studies, it was found that CSPG4 was also highly expressed in other malignant solid tumors, such as brain glioblastoma, triple negative breast cancer, head and neck squamous cell carcinoma and mesothelioma. CSPG4 was also highly expressed on the surface of capillaries in solid tumors. In contrast, CSPG4 is poorly expressed in normal tissues and critical organs. In addition, CSPG4 can regulate cell-matrix interaction, activate downstream ERK, FAK and other signaling pathways, and play an important role in tumor cell proliferation, migration, angiogenesis and metastasis.

Through genetically engineered T cells, chimeric antigen receptor—T cell therapy targets tumor cell surface antigens thereby killing tumor cells. Chimeric antigen receptor—T cell therapy has a greater targeting property than conventional surgery and chemoradiotherapy; compared with antibody therapy, it has stronger tumor-killing effect and better curative effect on malignant tumors. The success of this therapy in hematologic tumors also lays the foundation for its entry into the field of solid tumor therapy. In view of the high expression of CSPG4 on the surface of malignant solid tumors, its low expression in normal tissues, and its important role in tumorigenesis and metastasis, this makes it a good target for chimeric antigen receptor—T cell therapy. The CSPG4-targeting humanized chimeric antigen receptor was prepared and structurally optimized in this disclosure.

### SUMMARY OF THE INVENTION

The purpose of the present disclosure is to provide a CSPG4-targeting humanized chimeric antigen receptor (CAR), immune effector cells expressing the CAR, and applications thereof in cancer diagnosis, treatment and prevention.

Specifically, the present disclosure provides the following technical solutions:

In one aspect, the present disclosure provides a CSPG4-targeting humanized chimeric antigen receptor (CAR), the CAR comprises: an anti-CSPG4 binding domain, a hinge region, a transmembrane domain, and a signal transduction domain.

In one aspect, in the CAR of the present disclosure, wherein the anti-CSPG4 binding domain comprises an anti-CSPG4 antibody or antigen binding part comprising a heavy chain CDR selected from amino acid sequences shown in SEQ ID NOS: 5-7 or any variant thereof, and/or a light chain CDR selected from amino acid sequences shown in SEQ ID NOS: 10-12 or any variant thereof, characterized in that the anti-CSPG4 binding domain is humanized.

The CAR according to any of the preceding aspects, wherein the anti-C SPG4 binding domain is humanized means that, the variable region framework of the anti-CSPG4 binding domain is humanized.

The CAR according to any of the preceding aspects, wherein the variable region framework of the anti-CSPG4 binding domain is humanized means that, the heavy chain variable region framework comprises a heavy chain FR selected from amino acid sequences shown in SEQ ID NOS: 16-19 or any variant thereof; and/or, the light chain variable region framework comprises a light chain FR selected from amino acid sequences shown in SEQ ID NOS: 20-23 or any variant thereof.

The CAR according to any of the preceding aspects, wherein the variable region framework of the anti-CSPG4 binding domain is humanized means that, the heavy chain variable region framework comprises a heavy chain FR1 selected from the amino acid sequence shown in SEQ ID NO: 16 or any variant thereof, a heavy chain FR2 selected from the amino acid sequence shown in SEQ ID NO: 17 or any variant thereof, a heavy chain FR3 selected from the amino acid sequence shown in SEQ ID NO: 18 or any variant thereof, a heavy chain FR4 selected from the amino acid sequence shown in SEQ ID NO: 19 or any variant thereof; and/or a light chain FR1 selected from the amino acid sequence shown in SEQ ID NO: 20 or any variant thereof, a light chain FR2 selected from the amino acid sequence shown in SEQ ID NO: 21 or any variant thereof, a light chain FR3 selected from the amino acid sequence shown in SEQ ID NO: 22 or any variant thereof, a light chain FR4 selected from the amino acid sequence shown in SEQ ID NO: 23 or any variant thereof.

The CAR according to any of the preceding aspects, wherein the anti-CSPG4 binding domain comprises an anti-CSPG4 antibody or antigen binding part comprising a heavy chain CDR1 selected from the amino acid sequence shown in SEQ ID NO: 5 or any variant thereof, a heavy chain CDR2 selected from the amino acid sequence shown in SEQ ID NO: 6 or any variant thereof, a heavy chain CDR3 selected from the amino acid sequence shown in SEQ ID NO: 7 or any variant thereof; and/or a light chain CDR1 selected from the amino acid sequence shown in SEQ ID NO: 10 or any variant thereof, a light chain CDR2 selected from the amino acid sequence shown in SEQ ID NO: 11 or any variant thereof, a light chain CDR3 selected from the amino acid sequence shown in SEQ ID NO: 12 or any variant thereof.

The CAR according to any of the preceding aspects, wherein the anti-CSPG4 binding domain comprising an anti-CSPG4 antibody or antigen binding part comprising a heavy chain variable region sequence selected from the amino acid sequence shown in SEQ ID NO: 4 or any variant thereof; and/or a light chain variable region sequence selected from the amino acid sequence shown in SEQ ID NO: 9 or any variant thereof.

The CAR according to any of the preceding aspects, wherein the anti-CSPG4 binding domain comprises an anti-CSPG4 single-chain antibody (scFv), preferably the amino acid sequence of the anti-C SPG4 single-chain antibody is selected from amino acid sequences shown in SEQ ID NOS: 2, 14 or any variant thereof.

The CAR according to any of the preceding aspects, wherein the heavy and light chains of the anti-CSPG4 single-chain antibody are operably linked by a linker, preferably the linker comprises an amino acid sequence selected from the amino acid sequence shown in SEQ ID NO: 15 or any variant thereof.

The CAR according to any of the preceding aspects, wherein the transmembrane domain is selected from one or more of the α, β, ζ chains of TCR, CD3γ, CD3δ, CD3ε, CD3ζ, CD4, CD5, CD8α, CD8β, CD9, CD16, CD22, CD27, CD28, CD33, CD37, CD45, CD64, CD80, CD86, CD134, 4-1BB, CD152, CD154, PD-1, NKp44, NKp46 and NKG2D transmembrane domains; preferably, the transmembrane domain is selected from one or more of CD8α, CD8β, CD4, CD45, PD-1, CD154, CD28 transmembrane domains; preferably, the transmembrane domain is selected from one or more of CD8α, CD28 transmembrane domains; more preferably, the amino acid sequence of the transmembrane domain is selected from the amino acid sequence of SEQ ID NO: 27 or any variant thereof.

The CAR according to any of the preceding aspects, wherein the hinge region is selected from one or more of a CD8 extracellular hinge region, an IgG1 Fc CH2CH3 hinge region, an IgD hinge region, a CD28 extracellular hinge region, an IgG4 Fc CH2CH3 hinge region and a CD4 extracellular hinge region; preferably, the hinge region is a CD8α hinge region; more preferably, the amino acid sequence of the hinge region is selected from the amino acid sequence of SEQ ID NO: 25 or any variant thereof.

The CAR according to any of the preceding aspects, wherein the signal transduction domain is selected from one or more of TCRξ, FcRγ, FcRβ, CD3γ, CD3δ, CD3ε, CD5, CD22, CD79a, CD79b, CD278(ICOS), CD66d, DAP10, DAP12 and CD3ζ intracellular signal regions; preferably, the signal transduction domain is selected from a CD3ζ intracellular signal region; more preferably, the amino acid sequence of the signal transduction domain is selected from the amino acid sequence of SEQ ID NO: 33 or any variant thereof.

The CAR according to any of the preceding aspects, wherein the signal transduction domain further comprises one or more costimulatory domains; preferably, the costimulatory domain is selected from one or more of CARD11, CD2, CD7, CD27, CD28, CD30, CD40, CD54, CD83, OX40, 4-1BB, CD134, CD150, CD152, CD223, CD270, PD-L2, PD-L1, CD278, DAP10, DAP12, LAT, NKD2C, SLP76, TRIM, FcεRIγ, MyD88, 4-1BBL and 2B4 intracellular signal regions; preferably, the costimulatory domain is selected from 4-1BB, CD134, CD28 and OX40 intracellular signal regions; preferably, the costimulatory domain is selected from one or more of 4-1BB and CD28 intracellular signal domains; more preferably, the amino acid sequence of the costimulatory domain is selected from the amino acid sequences of SEQ ID NOS: 29, 31 or any variant thereof.

In one aspect, the present disclosure provides an isolated nucleic acid molecule comprising a polynucleotide sequence encoding the CAR of any of the preceding aspects; preferably, the nucleotide sequence of the nucleic acid molecule is selected from the nucleotide sequences of SEQ ID NOS: 1, 13 or any variant thereof.

In one aspect, the present disclosure provides a nucleic acid construct comprising the nucleic acid molecule of any of the previous aspects; preferably, the nucleic acid construct is a viral vector; more preferably, the viral vector is one or more of a retroviral vector, a lentiviral vector, an adenoviral vector, an adeno-associated viral vector.

In one aspect, the present disclosure provides a virus comprising the nucleic acid molecule of any of the preceding aspects, or comprising the nucleic acid construct of any of the preceding aspects; preferably, the virus is one or more of a retrovirus, a lentivirus, an adenovirus, an adeno-associated virus.

In one aspect, the present disclosure provides uses of the CAR of any of the preceding aspects, the nucleic acid molecule of any of the preceding aspects, the nucleic acid construct of any of the preceding aspects, the virus of any of the preceding aspects in the preparation of genetically modified immune cells of targeting CSPG4-expressing tumor cells.

In one aspect, the present disclosure provides an isolated host cell expressing the CAR of any of the preceding aspects, or comprising the isolated nucleic acid molecule of any of the preceding aspects, or comprising the nucleic acid construct of any of the preceding aspects, or comprising the virus of any of the preceding aspects; preferably, the host cell is a mammalian cell; more preferably, the host cell is one or more of a T cell, an NK cell, a γδT cell, an NKT cell, a macrophage or cell line, a PG13 cell line, a 293 cell line and cell lines derived therefrom; more preferably, the host cell is a T cell; most preferably, the host cell is a primarily cultured T cell.

The host cell according to any of the preceding aspects, the host cell further expresses other effector molecules including but not limited to one or more of cytokines, chemokines, another chimeric antigen receptor (CAR), chemokine receptors, siRNAs/shRNAs or sgRNAs knocking down or knocking out PD-1 expression or proteins blocking PD-L1, TCRs, and safety switches.

The host cell according to any of the preceding aspects, wherein the cytokine is selected from one or more of TNF-α, TNF-β, VEGF, TPO, NGF-β, PDGF, TGF-α, TGF-β, IGF-I, IGF-II, EPO, M-CSF, IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25 [symbol] LIF [symbol] FLT-3, interferon, angiostatin, thrombospondin, and endostatin.

The host cell according to any of the preceding aspects wherein the chemokine is selected from one or more of CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL3L3, CCL4, CCL4L1, CCL5, CCL6, CCL7, CCL8, CCL9, CX3CL1, CXCL1, CXCL10, CXCL11, CXCL12, CXCL13, CXCL14, CXCL15, CXCL16, CXCL17, CXCL2, CXCL3, CXCL4, CXCL5, CXCL6, CXCL7, CXCL9, CXCL8, XCL1, XCL2, FAM19A1, FAM19A2, FAM19A3, FAM19A4, and FAM19A5.

The host cell according to any of the preceding aspects wherein the chemokine receptor is selected from one or more of CCR1, CCR2, CCR3, CCR4, CCR5, CCR6, CCR7, CCR8, CCRL1, CXCR3, CXCR4, CXCR5, CXCR6, CXCR7, CXCR1, and CXCR2.

The host cell according to any of the preceding aspects wherein the safety switch is selected from one or more of HSVTK, VZVTK, iCaspase-9, iCaspase-1, iCaspase-8, truncated EGFR, and RQR8.

In one aspect, the present disclosure provides a pharmaceutical composition comprising one or more of the CAR of any of the preceding aspects, the nucleic acid molecule of the preceding aspects, the nucleic acid construct of the preceding aspects, the virus of the preceding aspects, the host cell of any of the preceding aspects, and a pharmaceutically acceptable carrier.

Uses of the CAR of any of the preceding aspects, the nucleic acid molecule of any of the preceding aspects, the nucleic acid construct of any of the preceding aspects, the virus of any of the preceding aspects, the host cell of any of the preceding aspects, or the pharmaceutical composition of any of the preceding aspects, in the preparation of a medicament; preferably, the medicament is used in diagnosing, treating or preventing cancer related diseases.

Preferably, the medicament is used in diagnosing, treating or preventing tumors expressing CSPG4; more preferably, the medicament is used in diagnosing, treating or preventing one or more selected from brain cancer, breast cancer, head and neck cancer, melanoma, mesothelioma; more preferably, the brain cancer is selected from one or more of brain glioblastoma, astrocytoma, meningioma, oligodendroglioma, glioma, the breast cancer is triple negative breast cancer, the head and neck cancer is head and neck squamous cell carcinoma.

Advantageous effects of the present disclosure include:

the CSPG4-targeting humanized chimeric antigen receptor of the present disclosure, as well as immune effector cells expressing the humanized chimeric antigen receptor, can reduce the generation of immune rejection and ensure better efficacy of the engineered CSPG4 chimeric antigen receptor—T cell therapy.

### BRIEF DESCRIPTION OF THE DRAWINGS

FIG. 1 shows the expression of CSPG4 in different tumor cell lines.

FIG. 2 shows the expression of CSPG4 in human brain glioma samples.

FIG. 3 shows the basic structure of CSPG4-targeting humanized chimeric antigen receptors.

FIG. 4 shows the expression of the CSPG4-targeting humanized chimeric antigen receptors on T cells.

FIG. 5A shows the killing effect on tumor cells by co-culture of CSPG4-targeting chimeric antigen receptor—T cells with tumor cells as measured by flow cytometry.

FIG. 5B shows the killing effect on tumor cells by co-culture of CSPG4-targeting chimeric antigen receptor—T cells with tumor cells, quantified by flow cytometry assay.

FIG. 6 shows cytokine levels upon tumor killed by the CSPG4-targeting chimeric antigen receptor—T cells.

FIG. 7 shows killing of brain gliomas in vivo by the CSPG4-targeting humanized chimeric antigen receptor—T cells.

FIG. 8 shows that the CSPG4-targeting humanized chimeric antigen receptor—T cells increase the survival rate of brain glioma model mice.

### DETAILED DESCRIPTION

### I. Definitions

In this disclosure, the scientific and technical terms used herein have the meanings commonly understood by those skilled in the art, unless otherwise specified. Also, as used herein, protein and nucleic acid chemistry, molecular biology, cell and tissue culture, microbiology, immunology, and laboratory procedures used herein are terms and routine procedures widely used in the corresponding fields. Meanwhile, in order to better understand the present disclosure, definitions and explanations of related terms are provided below.

It should also be understood that the terminology used herein is for the purpose of describing particular embodiments only and is not intended to be limiting.

As used herein and unless otherwise specified, the term “about” when referring to a measurable value such as an amount, time period, or the like, is meant to encompass variations of ±10%, more preferably ±5%, even more preferably ±1%, and still more preferably ±0.1% from the given value, so long as such variations are suitable for practicing the disclosed methods.

As used herein, the term “activation” refers to the state of a T cell that has been sufficiently stimulated to induce detectable cell proliferation. Activation may also be associated with induced cytokine production and detectable effector function. The terms “activated T cells” and the like refer to T cells that undergo cell division.

As used herein, the term “antibody” refers to an immunoglobulin molecule that specifically binds to an antigen. Antibodies can be intact immunoglobulins derived from natural sources or from recombinant sources, and can be immunoreactive portions of intact immunoglobulins. Antibodies are typically tetramers of immunoglobulin molecules. The antibodies of the present disclosure can exist in a variety of forms including, but not limited to: polyclonal antibodies, monoclonal antibodies, Fv, Fab and F(ab)₂, etc. as well as single chain antibodies and humanized antibodies (Harlow et al. 1999, using Antibodies: A Laboratory Manual, cold Spring Harbor Laboratory Press, NY; Harlow et al. 1989, Antibodies: A Laboratory Manual, Cold Spring Harbor, New York; Houston et al. 1988, Proc. Natl. Acad. Sci., USA 85: 5879-5883; Bird et al. 1988, Science 242: 423-426).

As used herein, an “antibody fragment” or “antigen binding fragment” of an antibody refers to any portion of a full-length antibody that is less than full-length, but that comprises at least a portion of the variable region (e.g. one or more CDR and/or one or more antibody binding sites) of the antibody that binds antigen, and thus retains binding specificity as well as at least a portion of the specific binding capacity of the full-length antibody. Thus, an antigen-binding fragment refers to an antibody fragment that comprises an antigen-binding portion that binds the same antigen as the antibody from which the antibody fragment is derived. Antibody fragments include antibody derivatives produced by enzymatic treatment of full-length antibodies, as well as synthetically produced derivatives, e.g. recombinantly produced derivatives. Antibodies include antibody fragments. Examples of antibody fragments include, but are not limited to, Fab, Fab′, F(ab′)2, single chain Fv (scFv), Fv, dsFv, diabodies, Fd and Fd′ fragments and other fragments, including modified fragments (see, e.g. Methods in Molecular Biology, Vol 207: Recombinant Antibodies for Cancer Therapy Methods and Protocols (2003); Chapter 1; p 3-25, Kipriyanov). The fragments may comprise multiple chains linked together, for example by disulfide bonds and/or by peptide linkers. Antibody fragments generally comprise at least or about 50 amino acids, and typically at least or about 200 amino acids. Antigen-binding fragments include any antibody fragment that, when inserted into an antibody framework (e.g. by displacement of the corresponding region), results in an antibody that immunospecifically binds (i.e. exhibits a Ka of at least or at least about 107-108M-1) to an antigen. A “functional fragment” or “analog of an anti-CSPG4 antibody” is a fragment or analog that prevents or substantially reduces the ability of the receptor to bind a ligand or initiate signal transduction. As used herein, functional fragments generally have the same meaning as “antibody fragments”, and in the case of antibodies, fragments that prevent or substantially reduce the ability of the receptor to bind a ligand or initiate signal transduction, e.g. Fv, Fab, F(ab′)2, and the like. An “Fv” fragment consists of a dimer of one heavy chain variable domain and one light chain variable domain formed by non-covalent association (VH-VL dimer). In this configuration, the three CDRs of each variable domain interact to determine a target binding site on the surface of the VH-VL dimer, as is the case with intact antibodies. The six CDRs together confer target binding specificity to the intact antibody. However, even a single variable domain (or half of an Fv comprising only three target-specific CDRs) may still have the ability to identify and bind targets. As used herein, the term “framework” or “FR” residues refers to those variable domain residues other than the hypervariable region residues defined herein as CDR residues.

As used herein, “monoclonal antibody” refers to a population of identical antibodies, meaning that each individual antibody molecule in the population of monoclonal antibodies is identical to another antibody molecule. This property is in contrast to the property of a polyclonal population of antibodies comprising antibodies having a plurality of different sequences. Monoclonal antibodies can be prepared by a number of well-known methods (Smith et al. (2004) J. Clin. Pathol. 57, 912-917; and Nelson et al. J Clin Pathol (2000), 53, 111-117). For example, monoclonal antibodies can be made by immortalizing B cells, for example by fusion with myeloma cells to produce hybridoma cell lines or by infecting B cells with a virus, such as EBV. Recombinant techniques can also be used to prepare antibodies from clonal populations of host cells in vitro by transforming host cells with plasmids carrying artificial sequences encoding the nucleotides of the antibodies.

An antibody “heavy chain”, as used herein, refers to the larger of the two types of polypeptide chains present in their naturally occurring conformations in all antibody molecules.

An antibody “light chain”, as used herein, refers to the smaller of the two types of polypeptide chains present in their naturally occurring conformations in all antibody molecules, and kappa and lambda light chains refer to the two major antibody light chain isotypes.

As used herein, the term “scFv” refers to a fusion protein comprising at least one variable region antibody fragment comprising a light chain and at least one variable region antibody fragment comprising a heavy chain, wherein the light and heavy chain variable regions are linked via a short, flexible polypeptide linker and are capable of being expressed as a single chain polypeptide, and wherein the scFv retains the specificity of the intact antibody from which it is derived. As used herein, unless specified, a scFv can have the VL and VH variable regions described in either order (e.g. relative to the N-terminus and C-terminus of the polypeptide), a scFv can comprise a VL-linker-VH or can comprise a VH-linker-VL.

As used herein, the term “gene synthesis” refers to production using recombinant DNA technology or obtained using synthetic DNA or amino acid sequence technology available and well known in the art.

As used herein, the term “antigen” or “Ag” is defined as a molecule that elicits an immune response that may involve antibody production, or activation of specific immunologically active cells, or both. The skilled artisan will appreciate that any macromolecule, including virtually all proteins or peptides, can be used as an antigen. In addition, the antigen may be derived from recombinant or genomic DNA. The skilled person will understand any DNA-which comprises a nucleotide sequence or part of a nucleotide sequence encoding a protein that elicits an immune response, thus encoding the term “antigen” as used herein. Furthermore, those skilled in the art will appreciate that the antigen need not be encoded solely by the full-length nucleotide sequence of the gene. It will be readily apparent that the present disclosure includes, but is not limited to, the use of partial nucleotide sequences of more than one gene, and that these nucleotide sequences are arranged in different combinations to elicit a desired immune response. Furthermore, the skilled person will appreciate that the antigen need not be encoded by a “gene” at all. It will be readily apparent that the antigen may be produced, synthesized or may be derived from a biological sample. Such biological samples may include, but are not limited to, tissue samples, tumor samples, cells, or biological fluids.

The term “anti-tumor effect”, as used herein, refers to a biological effect that may be clearly indicated by a reduction in tumor volume, a reduction in the number of tumor cells, a reduction in the number of metastases, an increase in life expectancy, or an improvement in various physiological symptoms associated with a cancerous condition. An “anti-tumor effect” may also be clearly indicated by the ability of the peptides, polynucleotides, cells and antibodies of the present disclosure to prevent the occurrence of a tumor at a first location.

The term “cancer” as used herein is defined as a disease characterized by rapid and uncontrolled growth of aberrant cells. Cancer cells can spread locally or through the blood stream and lymphatic system to other parts of the body. Examples of various cancers include, but are not limited to, brain cancer (e.g. astrocytoma, meningioma, oligodendroglioma, glioma, etc.), breast cancer (e.g. triple negative breast cancer), head and neck cancer (e.g. head and neck squamous cell carcinoma), melanoma, mesothelioma, etc.

As used herein, the term “co-stimulatory ligand” includes a molecule on an antigen-presenting cell (e.g. APC, dendritic cell, B cell, etc.) that specifically binds to an associated costimulatory molecule on a T cell, thereby providing a signal that mediates a T cell response, including but not limited to proliferation, activation, differentiation, etc. in addition to the primary signal provided by, for example, binding of the TCR/CD3 complex to a peptide-loaded MHC molecule. costimulatory ligands may include, but are not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin β receptor, 3/TR6, ILT3, ILT4, HVEM, agonists or antibodies that bind to the Toll ligand receptor, and ligands that specifically bind to CSPG4. costimulatory ligands also include, inter alia, antibodies that specifically bind to costimulatory molecules present on T cells, such as, but not limited to, antibodies that specifically bind to CD27, CD28, 4-1BB, OX40, CD30, CD40, PD-1, ICOS, lymphocyte function-associated antigen-1 (LFA-1), CD2, CD7, LIGHT, NKG2C, CSPG4 and CD83.

As used herein, the term “co-stimulatory molecule” refers to an associated binding partner on a T cell that specifically binds to a costimulatory ligand, thereby mediating a costimulatory response of the T cell, such as but not limited to proliferation, including but not limited to MHC-class I molecules, BTLA and Toll ligand receptors.

As used herein, the term “co-stimulatory signal” refers to a signal that, in combination with a primary signal, such as a TCR/CD3 junction, results in up-or down-regulation of T cell proliferation and/or key molecules.

“Encoding” refers to the use of a specific sequence of nucleotides in a polynucleotide such as a gene, cDNA, or mRNA as a template to synthesize the inherent properties of other polymers and macromolecules in biological processes that have any one of a defined sequence of nucleotides (i.e. rRNA, tRNA, and mRNA) or a defined sequence of amino acids and the biological properties resulting therefrom. Thus, a gene encodes a protein if transcription and translation of a mRNA corresponding to that gene produces the protein in a cell or other biological system. A nucleotide sequence that is identical to a mRNA sequence and is generally provided in the Sequence Listing, and a non-coding strand that serves as a template for transcription of a gene or cDNA, can both be referred to as encoding a protein or other product of that gene or cDNA.

As used herein, the term “endogenous” refers to any substance from or produced within an organism, cell, tissue or system.

As used herein, the term “exogenous” refers to any substance introduced from or produced outside of an organism, cell, tissue or system.

“expression” is defined as the transcription and/or translation of a particular nucleotide sequence driven by its promoter.

As used herein, the term “nucleic acid construct” refers to a vector comprising a recombinant polynucleotide comprising expression control sequences operably linked to a nucleotide sequence to be expressed. The nucleic acid construct comprises sufficient cis-acting elements for expression; other elements for expression may be supplied by the host cell or in an in vitro expression system. Nucleic acid constructs include all those known in the art, such as cosmids, plasmids (e.g. naked or contained in liposomes), and viruses (e.g. lentiviruses, retroviruses, adenoviruses, and adeno-associated viruses) that incorporate recombinant polynucleotides.

“Homologous” refers to sequence similarity or sequence identity between two polypeptides or between two nucleic acid molecules. When a position in two compared sequences is occupied by the same base or amino acid monomer subunit, for example, if a position in each of two DNA molecules is occupied by adenine, the molecules are homologous at that position. The percentage of homology between two sequences is a function of the number of matching or homologous positions shared by the two sequences divided by the number of positions compared×100. For example, two sequences are 60% homologous if 6 of the 10 positions in the two sequences are matched or homologous. By way of example, the DNA sequences ATTGCC and TATGGC share 50% homology. Typically, a comparison is made when two sequences are aligned to give maximum homology.

As used herein, the term “immunoglobulin” or “Ig” is defined as a class of proteins that function as antibodies. Antibodies expressed by B cells are sometimes referred to as BCR (B cell receptor) or antigen receptors. Five members included in this class of proteins are IgA, IgG, IgM, IgD and IgE. IgA are primary antibodies present in body secretions such as saliva, tears, breast milk, gastrointestinal secretions, and mucus secretions of the respiratory and genitourinary tracts. IgG is the most common circulating antibody. IgM is the predominant immunoglobulin produced in the primary immune response of most subjects. It is the most effective immunoglobulin in agglutination, complement fixation and other antibody responses and is important in combating bacteria and viruses. IgD is an immunoglobulin without known antibody function, but can be used as an antigen receptor. IgE are immunoglobulins that mediate immediate hypersensitivity after exposure to allergens by causing mediator release from mast cells and basophils.

“Isolated” means altered or removed from the natural state. For example, a nucleic acid or peptide that naturally occurs in a living animal is not “isolated”, but the same nucleic acid or peptide that is partially or completely separated from coexisting materials in its natural state is “isolated”. An isolated nucleic acid or protein can exist in substantially purified form or, for example, can exist in a non-natural environment, such as a host cell.

Unless otherwise specified, a “polynucleotide sequence encoding an amino acid sequence” includes all nucleotide sequences that are degenerate versions of each other and that encode the same amino acid sequence. The phrase nucleotide sequence encoding a protein or RNA may also include introns to the extent that the nucleotide sequence encoding the protein may include intron (s) in certain versions.

As used herein, the term “operably linked” refers to a functional linkage between a regulatory sequence and a heterologous nucleic acid sequence, which results in expression of the latter. For example, a first nucleic acid sequence is operably linked to a second nucleic acid sequence when the first nucleic acid sequence is in a functional relationship with the second nucleic acid sequence. For example, a promoter is operably linked to a coding sequence if the promoter affects the transcription or expression of the coding sequence. Generally, operably linked DNA sequences are contiguous, wherein two protein coding regions must be joined in the same reading frame.

The term “overexpressed” tumor antigen or “overexpression” of tumor antigen is intended to indicate an abnormal level of tumor antigen expression in cells from a disease area, such as a solid tumor within a particular tissue or organ of a patient, relative to the expression level of normal cells from the tissue or organ. Patients with solid tumors or hematological malignancies characterized by overexpression of tumor antigens can be determined by standard assays known in the art.

“Parenteral” administration of an immunogenic composition includes, for example, subcutaneous (s. c), intravenous (i. v.), intramuscular (i. m.), or intrasternal injection, or infusion techniques.

The terms “patient”, “subject”, “individual”, and the like are used interchangeably herein and refer to any animal or cell thereof, whether in vitro or in situ, that is amenable to the methods described herein. In some non-limiting embodiments, the patient, subject or individual is a human.

The term “polynucleotide” as used herein is defined as a strand of nucleotides. In addition, nucleic acids are multimers of nucleotides. Thus, nucleic acids and polynucleotides as used herein are interchangeable. Those skilled in the art have the general knowledge that nucleic acids are polynucleotides that can be hydrolyzed to monomeric “nucleotides”. Monomeric nucleotides can be hydrolyzed to nucleosides. Polynucleotides as used herein include, but are not limited to, all nucleic acid sequences obtained by any means available in the art, including, but not limited to, recombinant means, i.e. cloning nucleic acid sequences from recombinant libraries or cell genomes, using common cloning techniques and PCRTM, etc. and synthetic means.

As used herein, the terms “peptide”, “polypeptide”, and “protein” are used interchangeably and refer to a compound consisting of amino acid residues covalently linked by peptide bonds. The protein or peptide must comprise at least two amino acids, and there is no limitation on the maximum number of amino acids that can comprise the sequence of the protein or peptide. Polypeptides include any peptide or protein comprising two or more amino acids linked to each other by peptide bonds. As used herein, the term refers to short chains, which are also commonly referred to in the art as peptides, oligopeptides and oligomers; and longer chains, which are commonly referred to in the art as proteins, which are of many types. “Polypeptides” include, for example, biologically active fragments, substantially homologous polypeptides, oligopeptides, homodimers, heterodimers, variants of polypeptides, modified polypeptides, derivatives, analogs, fusion proteins, and the like. Polypeptides include natural peptides, recombinant peptides, synthetic peptides, or combinations thereof.

As used herein, the term “safety switch” refers to an engineered protein designed to prevent the potential toxicity of cell therapy or to otherwise prevent adverse effects. In some cases, safety switch protein expression is conditionally controlled to address safety issues of transplanted engineered cells that have permanently incorporated a gene encoding a safety switch protein into their genome. Such conditional regulation may be variable and may include control through small molecule-mediated post-translational activation and tissue-specific and/or on-time transcriptional regulation. The safety switch can mediate induction of apoptosis, inhibition of protein synthesis, DNA replication, growth arrest, transcriptional and post-transcriptional gene regulation, and/or antibody-mediated depletion. In some cases, the safety switch protein is activated by an exogenous molecule (e.g. a prodrug) that, when activated, triggers the treated cell to undergo apoptosis and/or cell death. Examples of safety switch proteins include, but are not limited to, HSVTK, VZVTK, iCaspase-9, iCaspase-1, iCaspase-8, truncated EGFR, RQR8, and the like. In this strategy, the prodrug administered in the event of an adverse event is activated by the suicide gene product and kills the transduced cell.

As used herein, the term “promoter” is defined as a DNA sequence required to initiate specific transcription of a polynucleotide sequence, recognized by, or directed by the synthetic machinery of a cell.

As used herein, the term “promoter/regulatory sequence” refers to a nucleic acid sequence required for expression of a gene product operably linked to a promoter/regulatory sequence. In some instances, the sequence may be a core promoter sequence, and in other instances, the sequence may also include enhancer sequences and other regulatory elements required for expression of the gene product. Promoter/regulatory sequences may, for example, be sequences that express a gene product in a tissue-specific manner.

A “constitutive” promoter is a nucleotide sequence that, when operably linked to a polynucleotide encoding or specifying a gene product, causes the gene product to be produced in a cell under most or all of the physiological conditions of the cell.

An “inducible” promoter is a nucleotide sequence that, when operably linked to a polynucleotide encoding or specifying a gene product, causes the gene product to be produced in a cell substantially only when an inducer corresponding to the promoter is present in the cell.

A “tissue-specific” promoter is a nucleotide sequence that, when operably linked to a polynucleotide encoding or defined by a gene, results in the production of a gene product in a cell essentially as long as the cell is of a tissue type corresponding to the promoter.

As used herein, “specific binding” or “immunospecifically binding” with respect to an antibody or antigen-binding fragment thereof is used interchangeably herein and refers to the ability of an antibody or antigen-binding fragment to form one or more non-covalent bonds with the same antigen through non-covalent interactions between the antibody and the antibody binding site of the antigen. The antigen may be an isolated antigen or present in a tumor cell. Typically, an antibody that immunospecifically binds (or specifically binds) to an antigen is one that binds to the antigen with an affinity constant Ka of about or 1×10⁷ M⁻¹ or 1×10⁸ M⁻¹ or more (or a dissociation constant (Kd) of 1×10⁻⁷M or 1×10⁻⁸ M or less). Affinity constants can be determined by standard kinetic methods for antibody reactions, e.g. immunoassays, surface plasmon resonance (SPR) (Rich and Myszka (2000) Curr. Opin. Biotechnol 11: 54; Englebienne (1998) Analyst. 123: 1599), isothermal titration calorimetry (ITC), or other kinetic interaction assays known in the art (see, e.g. Paul, ed. Fundamental Immunology, 2nd ed. Raven Press, New York, pages 332-336 (1989); see also U.S. Pat. No. 7,229,619, which describes an exemplary SPR and ITC method for calculating the binding affinity of an antibody). Instruments and methods for detecting and monitoring the rate of binding in real time are known and commercially available (see, BiaCore 2000, Biacore AB, Upsala, Sweden and GE Healthcare Life Sciences; Malmqvist (2000) Biochem. Soc. Trans. 27: 335).

As used herein, a “substantially purified” cell is a cell that is substantially free of other cell types. Substantially purified cells also refer to cells that have been separated from other cell types with which they are normally associated in their naturally occurring state. In some instances, a substantially purified cell population refers to a homogeneous cell population. In other instances, the term simply refers to a cell that has been separated from the cell with which it is normally associated in its native state. In some embodiments, the cells are cultured in vitro. In other embodiments, the cells are not cultured in vitro.

As used herein, the term “therapeutic” refers to both therapeutic and/or prophylactic. Therapeutic effects are achieved through inhibition, alleviation or eradication of a disease state.

As used herein, the term “therapeutically effective amount” refers to the amount of the subject compound that will elicit the biological or medical response of a tissue, system or subject that is being sought by the researcher, veterinarian, medical doctor or other clinician. The term “therapeutically effective amount” includes the amount of a compound that: when administered, it is sufficient to prevent the development of, or alleviate to some extent, one or more of the signs or symptoms of the disorder or disease being treated. The therapeutically effective amount will vary depending on the compound, the disease and its severity, and the age, weight, etc. of the subject to be treated.

“treating” a disease, as a term used herein, refers to reducing the frequency or severity of at least one sign or symptom of the disease or disorder that a subject is experiencing.

As used herein, the terms “transfected” or “transformed” or “transduced” refer to the process by which exogenous nucleic acid is transferred or introduced into a host cell. A “transfected” or “transformed” or “transduced” cell is a cell that has been transfected, transformed or transduced with an exogenous nucleic acid. Such cells include primary subject cells and progeny thereof.

As used herein, the term “vector” is a composition of matter that includes an isolated nucleic acid, and which can be used to deliver the isolated nucleic acid to the interior of a cell. Many vectors are known in the art, including but not limited to linear polynucleotides, polynucleotides related to ionic or amphipathic compounds, plasmids and viruses. Thus, the term “vector” includes an autonomously replicating plasmid or virus. The term should also be interpreted to include non-plasmid and non-viral compounds that facilitate transfer of nucleic acids into cells, such as, for example, polylysine compounds, liposomes, and the like. Examples of viral vectors include, but are not limited to, adenoviral vectors, adeno-associated viral vectors, retroviral vectors, and the like.

### II. Detailed Description of the Embodiments

In one aspect, the present disclosure provides humanized chimeric antigen receptors capable of targeting CSPG4, and immune effector cells expressing the CSPG4-targeting humanized chimeric antigen receptors; in specific aspects, the immune effector cells expressing the CSPG4-targeting humanized chimeric antigen receptor are T cells expressing the CSPG4-targeting humanized chimeric antigen receptor.

In one aspect, the CSPG4-targeting humanized chimeric antigen receptor (CAR) of the present disclosure comprises: an anti-CSPG4 binding domain, a hinge region, a transmembrane domain and a signal transduction domain, the anti-CSPG4 binding domain comprises an anti-CSPG4 antibody or antigen binding part comprising a heavy chain CDR selected from amino acid sequences shown in SEQ ID NOS: 5-7 or any variant thereof, and/or a light chain CDR selected from amino acid sequences shown in SEQ ID NOS: 10-12 or any variant thereof, the anti-CSPG4 binding domain is humanized.

The CAR according to any of the preceding aspects, wherein the anti-CSPG4 binding domain is humanized means that, the variable region framework of the anti-CSPG4 binding domain is humanized.

The CAR according to any of the preceding aspect, wherein the variable region framework of the anti-CSPG4 binding domain is humanized, meaning that the heavy chain variable region framework comprises one or more of the following amino acid residues: the amino acid at position 3 is Q, the amino acid at position 5 is V, the amino acid at position 6 is Q, the amino acid at position 9 is S, the amino acid at position 16 is A, the amino acid at position 17 is S, the amino acid at position 20 is V, the amino acid at position 38 is R, the amino acid at position 40 is A, the amino acid at position 43 is Q, the amino acid at position 46 is E, the amino acid at position 48 is M, the amino acid at position 63 is G, the amino acid at position 65 is T, the amino acid at position 69 is V, the amino acid at position 73 is D, the amino acid at position 84 is S, the amino acid at position 85 is S, and the amino acid at position 87 is K, the amino acid at position 88 is A, the amino acid at position 93 is V, and the amino acid at position 108 is L; and/or the light chain variable region framework comprises one or more of the following amino acid residues: the amino acid at position 3 is V, the amino acid at position 9 is L, the amino acid at position 10 is S, the amino acid at position 12 is P, the amino acid at position 18 is P, the amino acid at position 19 is A, the amino acid at position 21 is I, the amino acid at position 37 is L, the amino acid at position 40 is P, the amino acid at position 41 is G, the amino acid at position 42 is Q, the amino acid at position 45 is Q, the amino acid at position 58 is V, the amino acid at position 59 is D, the amino acid at position 63 is S, the amino acid at position 74 is K, the amino acid at position 76 is S, the amino acid at position 77 is R, and the amino acid at position 79 is E, the amino acid at position 80 is A, the amino acid at position 83 is V, the amino acid at position 85 is V, the amino acid at position 100 is Q, and the amino acid at position 108 is T.

The CAR according to any of the preceding aspects, wherein the variable region framework of the anti-CSPG4 binding domain is humanized means that, the heavy chain variable region framework comprises a heavy chain FR selected from amino acid sequences shown in SEQ ID NOS: 16-19 or any variant thereof; and/or, the light chain variable region framework comprises a light chain FR selected from amino acid sequences shown in SEQ ID NOS: 20-23 or any variant thereof.

The CAR according to any of the preceding aspects, wherein the variable region framework of the anti-CSPG4 binding domain is humanized means that, the heavy chain variable region framework comprises a heavy chain FR1 selected from the amino acid sequence shown in SEQ ID NO: 16 or any variant thereof, a heavy chain FR2 selected from the amino acid sequence shown in SEQ ID NO: 17 or any variant thereof, a heavy chain FR3 selected from the amino acid sequence shown in SEQ ID NO: 18 or any variant thereof, a heavy chain FR4 selected from the amino acid sequence shown in SEQ ID NO: 19 or any variant thereof; and/or a light chain FR1 selected from the amino acid sequence shown in SEQ ID NO: 20 or any variant thereof, a light chain FR2 selected from the amino acid sequence shown in SEQ ID NO: 21 or any variant thereof, a light chain FR3 selected from the amino acid sequence shown in SEQ ID NO: 22 or any variant thereof, a light chain FR4 selected from the amino acid sequence shown in SEQ ID NO: 23 or any variant thereof.

The CAR according to any of the preceding aspects, wherein the antibody or antigen binding part comprises a heavy chain CDR1 selected from the amino acid sequence shown in SEQ ID NO: 5 or any variant thereof, a heavy chain CDR2 selected from the amino acid sequence shown in SEQ ID NO: 6 or any variant thereof, a heavy chain CDR3 selected from the amino acid sequence shown in SEQ ID NO: 7 or any variant thereof; and/or a light chain CDR1 selected from the amino acid sequence shown in SEQ ID NO: 10 or any variant thereof, a light chain CDR2 selected from the amino acid sequence shown in SEQ ID NO: 11 or any variant thereof, a light chain CDR3 selected from the amino acid sequence shown in SEQ ID NO: 12 or any variant thereof.

The CAR according to any of the preceding aspects, wherein the anti-CSPG4 binding domain comprising an anti-CSPG4 antibody or antigen binding part comprising a heavy chain variable region sequence selected from the amino acid sequence shown in SEQ ID NO: 4 or any variant thereof; and/or a light chain variable region sequence selected from the amino acid sequence shown in SEQ ID NO: 9 or any variant thereof.

The CAR according to any of the preceding aspects, wherein the anti-CSPG4 binding domain comprises an anti-CSPG4 single-chain antibody (scFv) wherein the linker connecting the light chain and the heavy chain is humanized, preferably the linker comprises an amino acid sequence selected from the amino acid sequence shown in SEQ ID NO: 15 or any variant thereof.

The CAR according to any of the preceding aspects, wherein the anti-C SPG4 single-chain antibody is derived from a monoclonal antibody raised against a fragment of the CSPG4 protein.

The CAR according to any of the preceding aspects, wherein the amino acid sequence of the anti-CSPG4 single-chain antibody comprises amino acid sequences selected from SEQ ID NOS: 2, 14 or any variant thereof.

In one aspect, a humanized anti-CSPG4 binding domain has at least greater than 60%, 65%, 70%, 75%, 80%, 85%, 90%, 95%, 96%, 97%, 98%, 99% or more sequence identity to the anti-CSPG4 single-chain antibody portion of any of the preceding aspects.

The CAR according to any of the preceding aspects, the transmembrane domain is selected from one or more of the α, β, ζ chains of TCR, CD3γ, CD3δ, CD3ε, CD3ζ, CD4, CD5 ,CD8α, CD8β, CD9, CD16, CD22, CD27, CD28, CD33, CD37, CD45, CD64, CD80, CD86, CD134, 4-1BB, CD152, CD154, PD-1, NKp44, NKp46, and NKG2D transmembrane domains; preferably, the transmembrane domain is selected from one or more of CD8α, CD8β, CD4, CD45, PD-1, CD154, CD28 transmembrane domains; more preferably, the transmembrane domain is selected from one or more of CD8α, CD28 transmembrane domains; more preferably, the amino acid sequence of the transmembrane domain is selected from the amino acid sequence of SEQ ID NO: 27 or any variant thereof.

The CAR according to any of the preceding aspects, the hinge region is selected from one or more of a CD8 extracellular hinge region, an IgG1 Fc CH2CH3 hinge region, an IgD hinge region, a CD28 extracellular hinge region, an IgG4 Fc CH2CH3 hinge region and a CD4 extracellular hinge region; preferably, the hinge region is a CD8α hinge region; more preferably, the amino acid sequence of the hinge region is selected from the amino acid sequence of SEQ ID NO: 25 or any variant thereof.

The CAR according to any of the preceding aspects, the signal transduction domain is selected from one or more of TCRξ, FcRγ, FcRβ, CD3γ, CD3δ, CD3ε, CD5, CD22, CD79a, CD79b, CD278(ICOS), CD66d, DAP10, DAP12 and CD3ζ intracellular signal regions; preferably, the signal transduction domain is selected from a CD3ζ intracellular signal region; more preferably, the amino acid sequence of the signal transduction domain is selected from the amino acid sequence of SEQ ID NO: 33 or any variant thereof.

The CAR according to any of the preceding aspects, the signal transduction domain further comprises one or more costimulatory domains; preferably, the costimulatory domain is selected from one or more of the group consisting of CARD11, CD2, CD7, CD27, CD28, CD30, CD40, CD54, CD83, OX40, 4-1BB, CD134, CD150, CD152, CD223, CD270, PD-L2, PD-L1, CD278, DAP10, DAP12, LAT, NKD2C, SLP76, TRIM, FcεRIγ, MyD88, 41BBL, and 2B4 intracellular signal regions; more preferably, the costimulatory domain is selected from 4-1BB, CD134, CD28 and OX40 intracellular signal regions; more preferably, the costimulatory signal domain is selected from one or more of 4-1BB, CD28 intracellular signal domains; more preferably, the amino acid sequence of the costimulatory domain is selected from the amino acid sequences of SEQ ID NOS: 29, 31 or any variant thereof.

In another aspect, the present disclosure also provides a CSPG4-targeting single-chain antibody (scFV); in particular, the scFv is derived from monoclonal antibodies raised against fragments of the CSPG4 protein; more specifically, the amino acid sequence of the scFv comprises amino acid sequences selected from SEQ ID NOS: 2, 14 or any variant thereof.

In one aspect, the gene encoding CSPG4-targeting scFV comprises nucleotide sequences selected from SEQ ID NOS: 1, 13 or any variant thereof.

In one aspect, the present disclosure provides a nucleic acid molecule encoding a CAR or scFV according to any of the preceding aspects; preferably, the nucleotide sequences of the nucleic acid molecule encoding scFV are selected from SEQ ID NOS: 1, 13 or a nucleic acid molecule having at least greater than 60%, 65%, 70%, 75%, 80%, 85%, 90%, 95%, 96%, 97%, 98%, 99% or more sequence identity thereto.

In one aspect, the present disclosure provides a nucleic acid construct comprising the nucleic acid molecule of any of the previous aspects; preferably, the nucleic acid construct is a viral vector; more preferably, the viral vector is one or more of a retroviral vector, a lentiviral vector, an adenoviral vector.

In one aspect, the present disclosure provides a virus comprising the nucleic acid molecule of any of the preceding aspects, or comprising the nucleic acid construct of any of the preceding aspects; preferably, the virus is one or more of a retrovirus, a lentivirus, an adenovirus, an adeno-associated virus.

In one aspect, the present disclosure provides an isolated host cell expressing the CAR of any of the preceding aspects, or comprising the nucleic acid construct of any of the preceding aspects, or comprising the virus of any of the preceding aspects; preferably, the host cell is a mammalian cell; more preferably, the host cell is one or more of a T cell, an NK cell, a γδT cell, an NKT cell, a macrophage or cell line, a PG13 cell line, a 293 cell line and cell lines derived therefrom; more preferably, the host cell is a T cell; most preferably, the host cell is a primarily cultured T cell.

In one aspect, the host cell further expresses other effector molecules including but not limited to one or more of cytokines, chemokines, another chimeric antigen receptor (CAR), chemokine receptors, siRNA/shRNAs or sgRNAs knocking down or knocking out PD-1 expression or proteins blocking PD-L1, TCRs, and safety switches; preferably, the cytokine is selected from one or more of TNF-α, TNF-β, VEGF, TPO, NGF-β, PDGF, TGF-α, TGF-β, IGF-I, IGF-II, EPO, M-CSF, IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25 [symbol] LIF [symbol] FLT-3, interferon, angiostatin, thrombospondin, and endostatin; preferably, the chemokine is selected from one or more of CCL1, CCL11, CCL12, CCL13, CCL14, CCL15, CCL16, CCL17, CCL 18, CCL19, CCL2, CCL20, CCL21, CCL22, CCL23, CCL24, CCL25, CCL26, CCL27, CCL28, CCL3, CCL3L3, CCL4, CCL4L1, CCL5, CCL6, CCL7, CCL8, CCL9, CX3CL1, CXCL1, CXCL10, CXCL11, CXCL12, CXCL13, CXCL14, CXCL15, CXCL16, CXCL17, CXCL2, CXCL3, CXCL4, CXCL5, CXCL6, CXCL7, CXCL9, CXCL8, XCL1, XCL2, FAM19A1, FAM19A2, FAM19A3, FAM19A4 and FAM19A5. Preferably, the chemokine receptor is selected from one or more of CCR1, CCR2, CCR3, CCR4, CCR5, CCR6, CCR7, CCR8, CCRL1, CXCR3, CXCR4, CXCR5, CXCR6, CXCR7, CXCR1, CXCR2; preferably, the safety switch is selected from one or more of HSVTK, VZVTK, iCaspase-9, iCaspase-1, iCaspase-8, truncated EGFR and RQR8.

In one aspect, the present disclosure provides an immune effector cell that expresses the CSPG4-targeting chimeric antigen receptor; in particular, the present disclosure provides a T cell expressing the CSPG4-targeting chimeric antigen receptor (CAR-T); more specifically, the T cell is an activated T cell; more specifically, the activation of the T cells is accomplished by stimulation with CD3 and/or CD28 antibodies.

In one aspect, the present disclosure provides a pharmaceutical composition comprising one or more of the CAR, the nucleic acid molecule, the nucleic acid construct, the virus, the cell of any of the preceding aspects, and a pharmaceutically acceptable carrier.

It will be appreciated that therapeutic agents according to the embodiments will be administered with suitable pharmaceutically acceptable carriers, excipients, and other agents incorporated into formulations to provide improved transfer, delivery, tolerability, and the like. A large number of suitable formulations can be found in all pharmacopoeias known to pharmaceutical chemists: Remington's Pharmaceutical Science (15^{th} edition, Mack Publishing Company, Easton, Pa. (1975)), in particular Chapter 87 of Blaug, Seymour therein. These formulations include, for example, powders, pastes, ointments, gels, waxes, oils, lipids, lipid-containing (cationic or anionic) carriers (e.g. Lipofectin™), DNA conjugates, anhydrous syrups, oil-in-water and water-in-oil emulsions, emulsion polyethylene glycols (polyethylene glycols of various molecular weights), semi-solid gels, and semi-solid mixtures containing polyethylene glycol. Any of the foregoing mixtures may be suitable for the treatment or therapy according to the present disclosure, provided that the active ingredients in the formulation are not inactivated by the formulation and that the formulation is physiologically compatible and tolerates the route of administration.

As used herein, the term “pharmaceutically acceptable carrier” is intended to include any and all solvents, dispersion medium, coatings, antibacterial and antifungal agents, isotonic and absorption delaying agents, and the like, which are compatible with pharmaceutical administration. Suitable pharmaceutically acceptable carriers are described in the latest edition of Remington's Pharmaceutical Sciences, a standard reference text in the art, which is incorporated herein by reference. Preferred examples of such carriers or diluents include, but are not limited to, water, saline, ringer's solution, dextrose solution, and 1-10% human serum albumin. Liposomes and non-aqueous vehicles such as fixed oils can also be used. The use of such medium and agents for pharmaceutically active substances is well known in the art. Except insofar as any conventional medium or agent is incompatible with the antibody, its use in the compositions is contemplated.

Further, a pharmaceutical composition provided by the present disclosure can be used to treat diseases associated with inflammatory factors such as cancer, inflammatory diseases, autoimmune diseases, etc.

In one aspect, the use of a CAR, a nucleic acid molecule, a nucleic acid construct, a virus, a cell, a pharmaceutical composition of any of the preceding aspects in the preparation of a medicament for diagnosing, treating or preventing cancer.

In one embodiment, the medicament may be used as a therapeutic agent. Such agents will typically be used to diagnose, treat, ameliorate, and/or prevent a disease or pathology associated with aberrant CSPG4 expression, activity, and/or signal transduction in a subject. Treatment regimens can be performed using standard methods by identifying a subject, e.g. a person patient having (or at risk of or developing) a disease or disorder associated with aberrant CSPG4 expression, activity, and/or signal transduction, e.g. cancer or other neoplastic disorder. An antibody or antibody fragment specifically binding to a target antigen (such as CSPG4) is used as a chimeric antigen receptor (CAR) of the present disclosure, and the disclosed CAR is preferably prepared by an antibody having high specificity and high affinity to the target antigen (such as CSPG4), preferably, the prepared CAR is further used to genetically modify immune cells (such as T cells). By administering CAR and/or genetically modified immune cells of the present disclosure, expression, activity, and/or signal transduction functions of a target (e.g. CSPG4) can be eliminated, inhibited, or prevented. Binding of a target (e.g. CSPG4) to an endogenous ligand to which it naturally binds can be eliminated or inhibited or prevented by administration of a CAR and/or genetically modified immune cell of the present disclosure, e.g. CSPG4 expression, activity and/or signal transduction can be modulated, blocked, inhibited, reduced, antagonized, neutralized, or otherwise prevented following administration of a CAR and/or genetically modified immune cell of the present disclosure. In some embodiments, to treat a disease or disorder associated with aberrant CSPG4 expression, antibodies or antibody fragments of the anti-CSPG4 antibody heavy and light chain CDR can be prepared into the disclosed CAR and/or genetically modified immune cells for administration to a subject. In one embodiment, the disease or disorder associated with aberrant CSPG4 expression may be cancer.

For purposes of clarity and conciseness, features are described herein as part of the same or separate embodiments, however, it will be understood that the scope of the disclosure may include some embodiments having a combination of all or some of the features described.

### EXAMPLES

### Example 1: Detection of CSPG4 Expression in Tumor Cell Lines

To determine the expression of CSPG4 antigen in tumors, tumor cell lines of melanoma (WM-266-4 cell line), glioma (LN-229, U87-MG cell line) and breast cancer (MDA-MB-231 cell line) were first selected for detection. Meanwhile, in order to further verify the expression of CSPG4 in primary brain glioma, sections containing brain glioma tissues of multiple patients as well as normal tissues were detected by an immunohistochemical method using CSPG4 antibody.

Method: tumor cells were collected and washed three times with 1×PBS, then stained with CSPG4 antibody on ice for 30 min, after washing with 1×PBS, APC-conjugated secondary antibody was added, incubated at room temperatures for 20 min, washed with 1×PBS, and CSPG4 expression on tumor cells was detected by flow cytometry.

Immunohistochemical staining: paraffin sections were deparaffinized with xylene and rehydrated with alcohol, followed by antigen retrieval with sodium citrate, then blocked in serum for 1 h. The CSPG4 antibody was stained overnight at 4° C., and then incubated with horseradish peroxidase-coupled secondary antibody for 30 min. After washing, it was developed with DAB and then stained with hematoxylin. Sections were observed and photographed under a microscope after staining was completed.

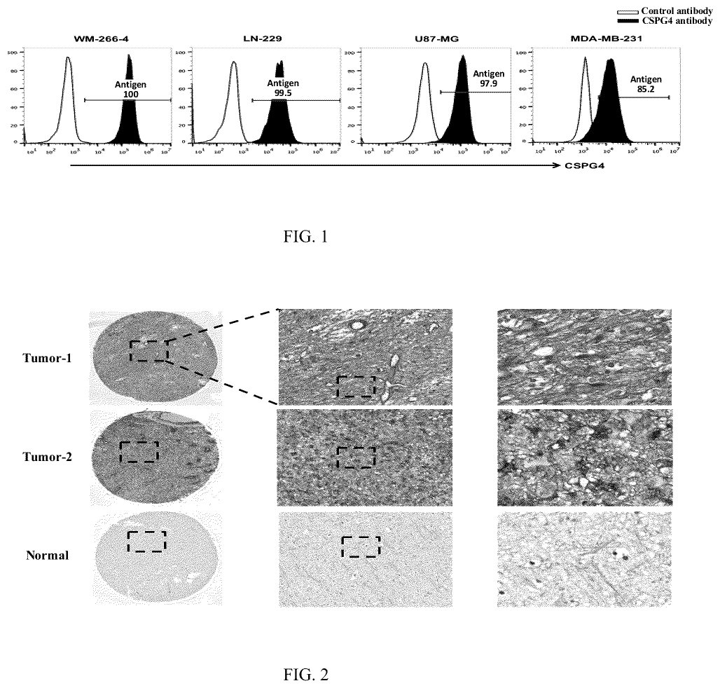

Results: as can be seen from FIG. 1, the detected tumor cells showed high expression of CSPG4 in all cases.

As can be seen from FIG. 2, the expression of CSPG4 was detected to varying degrees in the brain glioma samples of all patients, while the expression of CSPG4 was not found in normal brain tissues. This suggests that CSPG4 is a better target for brain glioma therapy.

### Example 2: Cell Culture

Cell lines used in the disclosure include 293T cells cultured in IMDM medium containing 10% FBS, 1% glutamax, 1% two types of antibiotics. WM-266-4, LN-229, U87-MG and MDA-MB-231 cell culture medium were 10% FBS in RPMI640 or DMEM. GFP positive cells were prepared by infection of retrovirus encoding GFP and tumor cell lines for mice were transfected with lentivirus or retrovirus encoding luciferase or GFP-firefly luciferase (GFP-FFLuc).

### Example 3: Construction of Chimeric Antigen Receptors

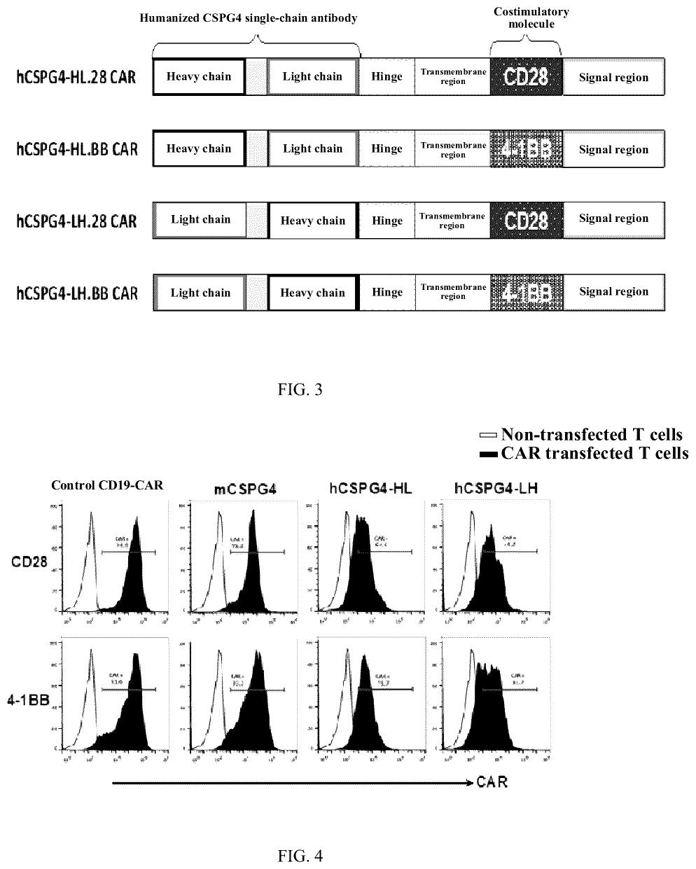

Existing chimeric antigen receptors for CSPG4 are derived from the mouse monoclonal antibody 763.74 (WO2015080981), however, considering that mouse antibodies or chimeric antigen receptors will elicit “human anti-mouse” antibodies in human bodies, thereby greatly diminishing the therapeutic efficacy of the antibody or chimeric antigen receptor—T cells. In view of the above considerations, the present disclosure humanizes the chimeric antigen receptor derived from the antibody 763.74, and also optimizes the structure of the original chimeric antigen receptor (the original mouse chimeric antigen receptor (mCSPG4) is a heavy chain-light chain structure, and the humanized chimeric antigen receptor has two structures of a heavy chain-light chain (hCSPG4-HL) and a light chain-heavy chain (hCSPG4-LH), see FIG. 3), and the present disclosure also makes a functional comparison with the mouse chimeric antigen receptor.

The humanized anti-CSPG4 chimeric antigen receptors of the present disclosure consists of a single-chain antibody region, a CD8α hinge region, a CD8α transmembrane region, a CD28 or 4-1BB costimulatory factor region, and a CD3ζ signal transduction region. The single-chain antibody region of the chimeric antigen receptor is derived from the humanization of the sequence of the antibody 763.74. Chimeric antigen receptors are generated by gene synthesis and then cloned into retroviral vectors. The correctness of the sequence was confirmed by sequencing after cloning.

TABLE 1
Sequence List:

SEQ ID NO: 1: nucleic acid sequence of the humanized CSPG4-HL (hCSPG4-HL) single-chain
antibody region
ATGGAGTTTGGGCTGAGCTGGCTTTTTCTTGTGGCTATTTTAAAAGGTGTCCAGTGCT
CTAGACAGGTTCAGCTGGTGCAGTCTGGCAGCGAGCTGAAGAAACCTGGCGCCTCT
GTGAAGGTGTCCTGCAAGGCTAGCGGCTACACATTCACCGACTACAGCATGCACTGG
GTCCGAAAGGCCCCTGGACAGGGACTTGAATGGATGGGCTGGATCAACACCGCCAC
AGGCGAGCCTACCTACGCCGATGGCTTCACAGGCAGATTCGTGATCAGCCTGGACAC
CAGCGCCAGAACCGTGTACCTGCAGATCAGCTCTCTGAAGGCCGAGGATACCGCCGT
GTACTTCTGCTTCAGCTACTACGACTACTGGGGCCAGGGCACCCTGGTCACAGTTTCT
TCTGGTGGTGGTGGTTCTGGTGGTGGTGGTTCTGGCGGCGGCGGCTCCGGTGGTGGT
GGATCCGACATCGTGCTGACACAGAGCCCTCTGAGCCTGCCTGTTACACCTGGCGAG
CCTGCCAGCATCAGCTGTAGAGCCAGCCAGACCATCTACAAGAACCTGCACTGGTAT
CTGCAGAAGCCCGGCCAGTCTCCTCAGCTGCTGATTAAGTACGGCAGCGACAGCATC
TCCGGCGTGCCAGATAGATTCAGCGGCTCTGGCTCTGGCACCGACTACACCCTGAAG
ATCAGCAGAGTGGAAGCCGAGGACGTGGGCGTGTACTACTGTCTGCAAGGCTACAG
CACCCCTTGGACATTCGGCCAGGGCACCAAGCTGGAAATCAAG
SEQ ID NO: 2: protein sequence of the humanized CSPG4-HL (hCSPG4-HL) single-chain
antibody region
MEFGLSWLFLVAILKGVQCSRQVQLVQSGSELKKPGASVKVSCKASGYTFTDYSMHWV
RKAPGQGLEWMGWINTATGEPTYADGFTGRFVISLDTSARTVYLQISSLKAEDTAVYFCF
SYYDYWGQGTLVTVSSGGGGSGGGGSGGGGSGGGGSDIVLTQSPLSLPVTPGEPASISCR
ASQTIYKNLHWYLQKPGQSPQLLIKYGSDSISGVPDRFSGSGSGTDYTLKISRVEAEDVG
VYYCLQGYSTPWTFGQGTKLEIK
SEQ ID NO: 3: nucleic acid sequence of the humanized CSPG4 (hCSPG4) single-chain
antibody heavy chain
CAGGTTCAGCTGGTGCAGTCTGGCAGCGAGCTGAAGAAACCTGGCGCCTCTGTGAA
GGTGTCCTGCAAGGCTAGCGGCTACACATTCACCGACTACAGCATGCACTGGGTCCG
AAAGGCCCCTGGACAGGGACTTGAATGGATGGGCTGGATCAACACCGCCACAGGCG
AGCCTACCTACGCCGATGGCTTCACAGGCAGATTCGTGATCAGCCTGGACACCAGCG
CCAGAACCGTGTACCTGCAGATCAGCTCTCTGAAGGCCGAGGATACCGCCGTGTACT
TCTGCTTCAGCTACTACGACTACTGGGGCCAGGGCACCCTGGTCACAGTTTCTTCT
SEQ ID NO: 4: protein sequence of the humanized CSPG4 (hCSPG4) single-chain antibody
heavy chain
QVQLVQSGSELKKPGASVKVSCKASGYTFTDYSMHWVRKAPGQGLEWMGWINTATGE
PTYADGFTGRFVISLDTSARTVYLQISSLKAEDTAVYFCFSYYDYWGQGTLVTVSS
SEQ ID NO: 5: protein sequence of the humanized CSPG4 (hCSPG4) single-chain antibody
heavy chain CDR1
GYTFTDYS
SEQ ID NO: 6: protein sequence of the humanized CSPG4 (hCSPG4) single-chain antibody
heavy chain CDR2
INTATGEP
SEQ ID NO: 7: protein sequence of the humanized CSPG4 (hCSPG4) single-chain antibody
heavy chain CDR3
FSYYDY
SEQ ID NO: 8: nucleic acid sequence of the humanized CSPG4 (hCSPG4) single-chain
antibody light chain
GACATCGTGCTGACACAGAGCCCTCTGAGCCTGCCTGTTACACCTGGCGAGCCTGCC
AGCATCAGCTGTAGAGCCAGCCAGACCATCTACAAGAACCTGCACTGGTATCTGCAG
AAGCCCGGCCAGTCTCCTCAGCTGCTGATTAAGTACGGCAGCGACAGCATCTCCGGC
GTGCCAGATAGATTCAGCGGCTCTGGCTCTGGCACCGACTACACCCTGAAGATCAGC
AGAGTGGAAGCCGAGGACGTGGGCGTGTACTACTGTCTGCAAGGCTACAGCACCCC
TTGGACATTCGGCCAGGGCACCAAGCTGGAAATCAAG
SEQ ID NO: 9: protein sequence of the humanized CSPG4 (hCSPG4) single-chain antibody
light chain
DIVLTQSPLSLPVTPGEPASISCRASQTIYKNLHWYLQKPGQSPQLLIKYGSDSISGVPDRF
SGSGSGTDYTLKISRVEAEDVGVYYCLQGYSTPWTFGQGTKLEIK
SEQ ID NO: 10: protein sequence of the humanized CSPG4 (hCSPG4) single-chain antibody
light chain CDR1
RASQTIYKNLH
SEQ ID NO: 11: protein sequence of the humanized CSPG4 (hCSPG4) single-chain antibody
light chain CDR2
YGSDSIS
SEQ ID NO: 12: protein sequence of the humanized CSPG4 (hCSPG4) single-chain antibody
light chain CDR3
LQGYSTPWT
SEQ ID NO: 13: nucleic acid sequence of the humanized CSPG4-LH (hCSPG4-LH)
single-chain antibody region
ATGGAGTTTGGGCTGAGCTGGCTTTTTCTTGTGGCTATTTTAAAAGGTGTCCAGTGCT
CTAGAGACATCGTGCTGACACAGAGCCCTCTGAGCCTGCCTGTTACACCTGGCGAGC
CTGCCAGCATCAGCTGTAGAGCCAGCCAGACCATCTACAAGAACCTGCACTGGTATC
TGCAGAAGCCCGGCCAGTCTCCTCAGCTGCTGATTAAGTACGGCAGCGACAGCATCT
CCGGCGTGCCAGATAGATTCAGCGGCTCTGGCTCTGGCACCGACTACACCCTGAAGA
TCAGCAGAGTGGAAGCCGAGGACGTGGGCGTGTACTACTGTCTGCAAGGCTACAGC
ACCCCTTGGACATTCGGCCAGGGCACCAAGCTGGAAATCAAGGGTGGTGGTGGTTCT
GGTGGTGGTGGTTCTGGCGGCGGCGGCTCCGGTGGTGGTGGATCCCAGGTTCAGCTG
GTGCAGTCTGGCAGCGAGCTGAAGAAACCTGGCGCCTCTGTGAAGGTGTCCTGCAA
GGCTAGCGGCTACACATTCACCGACTACAGCATGCACTGGGTCCGAAAGGCCCCTGG
ACAGGGACTTGAATGGATGGGCTGGATCAACACCGCCACAGGCGAGCCTACCTACGC
CGATGGCTTCACAGGCAGATTCGTGATCAGCCTGGACACCAGCGCCAGAACCGTGTA
CCTGCAGATCAGCTCTCTGAAGGCCGAGGATACCGCCGTGTACTTCTGCTTCAGCTAC
TACGACTACTGGGGCCAGGGCACCCTGGTCACAGTTTCTTCT
SEQ ID NO: 14: protein sequence of the humanized CSPG4-LH (hCSPG4-LH) single-chain
antibody region
MEFGLSWLFLVAILKGVQCSRDIVLTQSPLSLPVTPGEPASISCRASQTIYKNLHWYLQKP
GQSPQLLIKYGSDSISGVPDRFSGSGSGTDYTLKISRVEAEDVGVYYCLQGYSTPWTFGQ
GTKLEIKGGGGSGGGGSGGGGSGGGGSQVQLVQSGSELKKPGASVKVSCKASGYTFTD
YSMHWVRKAPGQGLEWMGWINTATGEPTYADGFTGRFVISLDTSARTVYLQISSLKAED
TAVYFCFSYYDYWGQGTLVTVSS
SEQ ID NO: 15: protein sequence of the humanized CSPG4 (hCSPG4) single-chain antibody
linker
GGGGSGGGGSGGGGSGGGGS
SEQ ID NO: 16: protein sequence of the humanized CSPG4 (hCSPG4) single-chain antibody
heavy chain FR1
QVQLVQSGSELKKPGASVKVSCKAS
SEQ ID NO: 17: protein sequence of the humanized CSPG4 (hCSPG4) single-chain antibody
heavy chain FR2
MHWVRKAPGQGLEWMGW
SEQ ID NO: 18: protein sequence of the humanized CSPG4L (hCSPG4) single-chain antibody
heavy chain FR3
TYADGFTGRFVISLDTSARTVYLQISSLKAEDTAVYFC
SEQ ID NO: 19: protein sequence of the humanized CSPG4 (hCSPG4) single-chain antibody
heavy chain FR4
WGQGTLVTVSS
SEQ ID NO: 20: protein sequence of the humanized CSPG4 (hCSPG4) single-chain antibody
light chain FR1
DIVLTQSPLSLPVTPGEPASISC
SEQ ID NO: 21: protein sequence of the humanized CSPG4 (hCSPG4) single-chain antibody
light chain FR2
WYLQKPGQSPQLLIK
SEQ ID NO: 22: protein sequence of the humanized CSPG4 (hCSPG4) single-chain antibody
light chain FR3
GVPDRFSGSGSGTDYTLKISRVEAEDVGVYYC
SEQ ID NO: 23: protein sequence of the humanized CSPG4 (hCSPG4) single-chain antibody
light chain FR4
FGQGTKLEIK
SEQ ID NO: 24: nucleic acid sequence of CD8α hinge region
ACCACGACGCCAGCGCCGCGACCACCAACACCGGCGCCCACCATCGCGTCGCAGCC
CCTGTCCCTGCGCCCAGAGGCGTGCCGGCCAGCGGCGGGGGGCGCAGTGCACACGA
GGGGGCTGGACTTCGCCTGTGAT
SEQ ID NO: 25: protein sequence of CD8α hinge region
TTTPAPRPPTPAPTIASQPLSLRPEACRPAAGGAVHTRGLDFACD
SEQ ID NO: 26: nucleic acid sequence of CD8α transmembrane region
ATCTACATCTGGGCGCCCTTGGCCGGGACTTGTGGGGTCCTTCTCCTGTCACTGGTTA
TCACCCTTTACTGC
SEQ ID NO: 27: protein sequence of CD8α transmembrane region
IYIWAPLAGTCGVLLLSLVITLYC
SEQ ID NO: 28: nucleic acid sequence of CD28 costimulatory factor
AGGAGTAAGAGGAGCAGGCTCCTGCACAGTGACTACATGAACATGACTCCCCGCCGC
CCCGGGCCCACCCGCAAGCATTACCAGCCCTATGCCCCACCACGCGACTTCGCAGCC
TATCGCTCC
SEQ ID NO: 29: protein sequence of CD28 costimulatory factor
RSKRSRLLHSDYMNMTPRRPGPTRKHYQPYAPPRDFAAYRS
SEQ ID NO: 30: nucleic acid sequence of 4-1BB costimulatory factor
AAACGGGGCAGAAAGAAACTCCTGTATATATTCAAACAACCATTTATGAGACCAGTAC
AAACTACTCAAGAGGAAGATGGCTGTAGCTGCCGATTTCCAGAAGAAGAAGAAGGA
GGATGTGAACTG
SEQ ID NO: 31: protein sequence of 4-1BB costimulatory factor
KRGRKKLLYIFKQPFMRPVQTTQEEDGCSCRFPEEEEGGCEL
SEQ ID NO: 32: nucleic acid sequence of CD3ζ signal transduction region
AGAGTGAAGTTCAGCAGGAGCGCAGACGCCCCCGCGTACCAGCAGGGCCAGAACCA
GCTCTATAACGAGCTCAATCTAGGACGAAGAGAGGAGTACGATGTTTTGGACAAGAG
ACGTGGCCGGGACCCTGAGATGGGGGGAAAGCCGAGAAGGAAGAACCCTCAGGAA
GGCCTGTACAATGAACTGCAGAAAGATAAGATGGCGGAGGCCTACAGTGAGATTGGG
ATGAAAGGCGAGCGCCGGAGGGGCAAGGGGCACGATGGCCTTTACCAGGGTCTCAG
TACAGCCACCAAGGACACCTACGACGCCCTTCACATGCAGGCCCTGCCCCCTCGC
SEQ ID NO: 33: protein sequence of CD3ζ signal transduction region
RVKFSRSADAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR
SEQ ID NO. 34: nucleic acid sequence of CD19-CAR
ATGGAGTTTGGGCTGAGCTGGCTTTTTCTTGTGGCTATTTTAAAAGGTGTCCAGTGCT
CTAGAGACATCCAGATGACACAGACTACATCCTCCCTGTCTGCCTCTCTGGGAGACA
GAGTCACCATCAGTTGCAGGGCAAGTCAGGACATTAGTAAATATTTAAATTGGTATCA
GCAGAAACCAGATGGAACTGTTAAACTCCTGATCTACCATACATCAAGATTACACTCA
GGAGTCCCATCAAGGTTCAGTGGCAGTGGGTCTGGAACAGATTATTCTCTCACCATTA
GCAACCTGGAGCAAGAAGATATTGCCACTTACTTTTGCCAACAGGGTAATACGCTTCC
GTACACGTTCGGAGGGGGGACCAAGCTGGAGCTGAAACGTGGTGGTGGTGGTTCTG
GTGGTGGTGGTTCTGGCGGCGGCGGCTCCGGTGGTGGTGGATCCGAGGTGCAGCTGC
AGCAGTCTGGACCTGGCCTGGTGGCGCCCTCACAGAGCCTGTCCGTCACATGCACTG
TCTCAGGGGTCTCATTACCCGACTATGGTGTAAGCTGGATTCGCCAGCCTCCACGAAA
GGGTCTGGAGTGGCTGGGAGTAATATGGGGTAGTGAAACCACATACTATAATTCAGCT
CTCAAATCCAGACTGACCATCATCAAGGACAACTCCAAGAGCCAAGTTTTCTTAAAA
ATGAACAGTCTGCAAACTGATGACACAGCCATTTACTACTGTGCCAAACATTATTACT
ACGGTGGTAGCTATGCTATGGACTACTGGGGCCAAGGGACCTCAGTCACCGTCTCCTC
AACGCGTACCACGACGCCAGCGCCGCGACCACCAACACCGGCGCCCACCATCGCGT
CGCAGCCCCTGTCCCTGCGCCCAGAGGCGTGCCGGCCAGCGGCGGGGGGCGCAGTG
CACACGAGGGGGCTGGACTTCGCCTGTGATATCTACATCTGGGCGCCCTTGGCCGGG
ACTTGTGGGGTCCTTCTCCTGTCACTGGTTATCACCCTTTACTGCAGGAGTAAGAGGA
GCAGGCTCCTGCACAGTGACTACATGAACATGACTCCCCGCCGCCCCGGGCCCACCC
GCAAGCATTACCAGCCCTATGCCCCACCACGCGACTTCGCAGCCTATCGCTCCAGAGT
GAAGTTCAGCAGGAGCGCAGACGCCCCCGCGTACCAGCAGGGCCAGAACCAGCTCT
ATAACGAGCTCAATCTAGGACGAAGAGAGGAGTACGATGTTTTGGACAAGAGACGTG
GCCGGGACCCTGAGATGGGGGGAAAGCCGAGAAGGAAGAACCCTCAGGAAGGCCT
GTACAATGAACTGCAGAAAGATAAGATGGCGGAGGCCTACAGTGAGATTGGGATGAA
AGGCGAGCGCCGGAGGGGCAAGGGGCACGATGGCCTTTACCAGGGTCTCAGTACAG
CCACCAAGGACACCTACGACGCCCTTCACATGCAGGCCCTGCCCCCTCGC
SEQ ID NO. 35: protein sequence of CD19-CAR
MEFGLSWLFLVAILKGVQCSRDIQMTQTTSSLSASLGDRVTISCRASQDISKYLNWYQQK
PDGTVKLLIYHTSRLHSGVPSRFSGSGSGTDYSLTISNLEQEDIATYFCQQGNTLPYTFGG
GTKLELKRGGGGSGGGGSGGGGSGGGGSEVQLQQSGPGLVAPSQSLSVTCTVSGVSLPD
YGVSWIRQPPRKGLEWLGVIWGSETTYYNSALKSRLTIIKDNSKSQVFLKMNSLQTDDT
AIYYCAKHYYYGGSYAMDYWGQGTSVTVSSTRTTTPAPRPPTPAPTIASQPLSLRPEACR
PAAGGAVHTRGLDFACDIYIWAPLAGTCGVLLLSLVITLYCRSKRSRLLHSDYMNMTPRR
PGPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLS
TATKDTYDALHMQALPPR

### Example 4: Preparation of the Retroviruses for Chimeric Antigen Receptors

Murine leukemia retrovirus (MMLV) is prepared by 293T cells transiently transfected using a three-plasmid system (GFS-CAR, MMLV-gag-pol and RV-RD). CD19-CAR was used as a control for subsequent studies, while murine (mCSPG4) and humanized (hCSPG4-HL, hCSPG4-LH) CARs were the target CARs to be tested. At 48 and 72 h after transfection, 293T cell supernatants were harvested for infection of T cells. Among them, the nucleotide sequence of CD19-CAR is shown in SEQ ID NO: 34, and the amino acid sequence thereof is shown in SEQ ID NO: 35.

### Example 5: Preparation of T Cells Expressing CSPG4-targeting Humanized Chimeric Antigen Receptor

### 1. Infection and Amplification of T Cells

Blood donated from healthy individuals was used to isolate peripheral blood mononuclear cells. Mononuclear cells were isolated using lymphocyte density gradient separation. After activation of the isolated mononuclear cells by antibodies to CD3 and CD28, T cells were infected with the harvested retroviruses. The chimeric antigen receptor transfected T cells were then amplified for detection of chimeric antigen receptor expression and tumor killing.

2. Detection of the Expression of the CSPG4 Chimeric Antigen Receptors

A portion of amplified chimeric antigen receptor—T cells were harvested and the expression of CSPG4 chimeric antigen receptors were detected using biotin-labeled protein L, and the secondary antibody was phycoerythrin-labeled avidin. Flow cytometry was used to detect the expression of the chimeric antigen receptors on T cells.

Results: as can be seen from FIG. 4, the murine (mCSPG4) or humanized (hCSPG4-HL or hCSPG4-LH) CSPG4 chimeric antigen receptors (CD28 or 4-1BB costimulatory domain) can be expressed on human T cells. The expression of murine chimeric antigen receptors was slightly higher than that of humanized receptors, mainly due to the decreased binding of the staining reagent caused by the humanization of the chimeric antigen receptor.

### Example 6: Killing Effect of CSPG4 Humanized Chimeric Antigen Receptor—T Cells on Tumor Cells (Cell Co-culture)

To compare whether the engineered chimeric antigen receptor of the present disclosure can mediate T cell killing of tumor cells highly expressing CSPG4 as the original murine receptor, co-culture experiments were designed to test the killing of various tumor cells by three chimeric antigen receptor—T cells (mCSPG4, hCSPG4-HL, hCSPG4-LH). After tumor cell attachment, T cells expressing control (chimeric antigen receptor against CD19, CD19-CAR), murine and two humanized CSPG4 chimeric antigen receptors were added, and the killing thereof on melanoma, glioma and breast cancer cells was detected by flow cytometry.

Method: GFP-labeled tumor cells were seeded at a density of 1-2×10⁵ per well in 24-well plates, and 24 h later corresponding numbers of T cells were added at different ratios. After 7 days the cells in the wells were harvested and the remaining cells were detected by flow cytometry. T cells were detected using CD3, and tumor cells were detected using GFP.

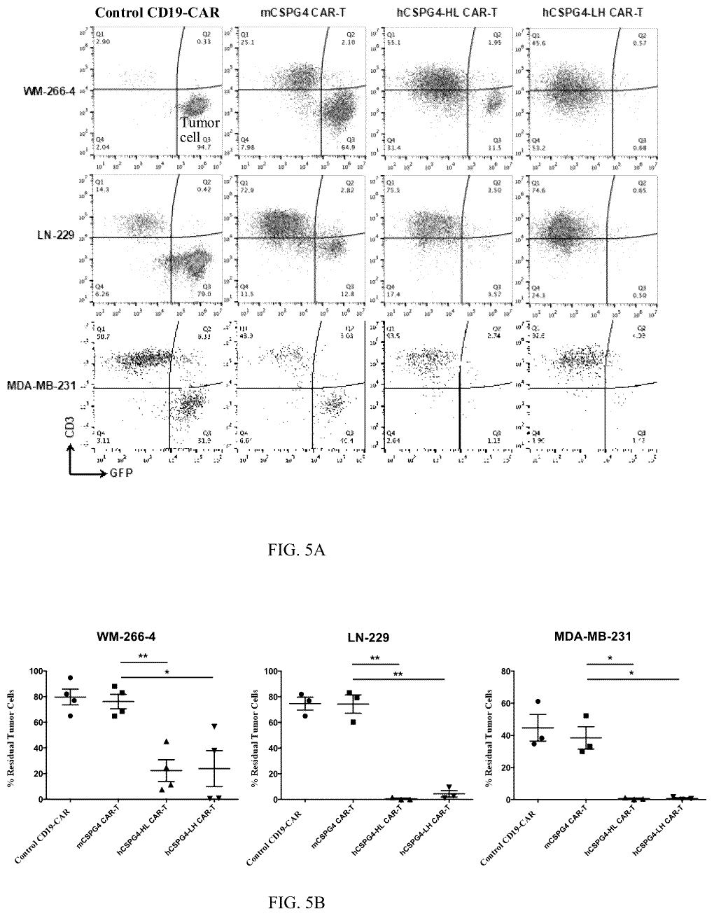

Results: as shown in FIGS. 5A and 5B, murine CSPG4 chimeric antigen receptor—T cells show some killing effect on three tumor cells. In contrast, the T cells of the humanized CSPG4 chimeric antigen receptors of the present disclosure can kill tumor cells more effectively in co-culture with three tumor cells, and in particular, the structurally-optimized humanized CSPG4 chimeric antigen receptor (hCSPG4-LH) can completely eliminate tumor cells.

### Example 7: Effect of CSPG4 Humanized Chimeric Antigen Receptor—T Cells on Cytokine Release During Tumor Cell Killing

Method: enzyme-linked immunosorbent assay (ELISA)

Supernatants were harvested 24 h after the addition of T cells in experiments where tumor cells were co-cultured with chimeric antigen receptor—T cells. Cytokines were detected in the supernatants using an ELISA kit.

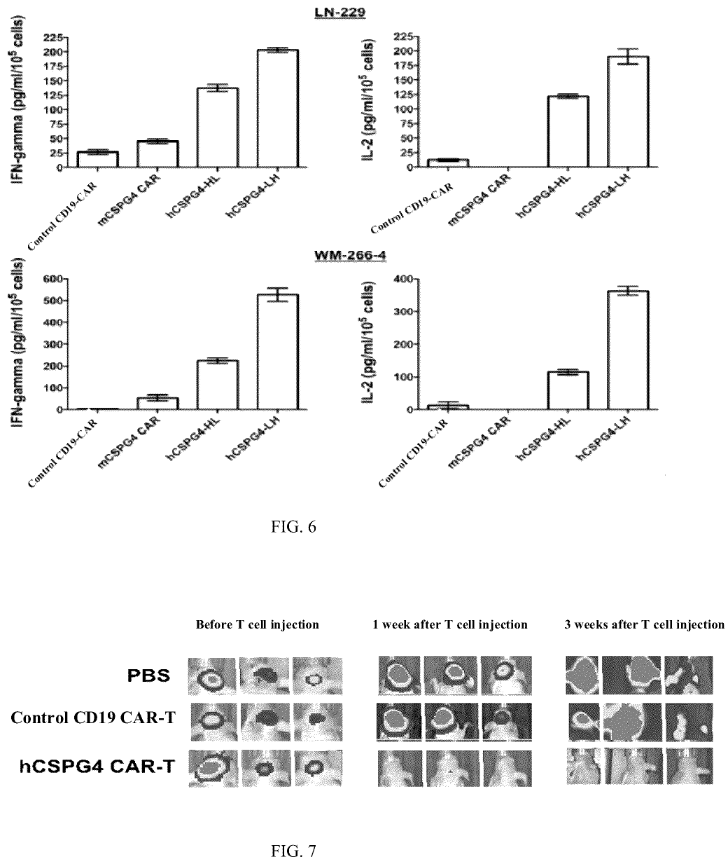

Results: the results are consistent with that of the cytotoxicity experiments, it can be seen from FIG. 6 that the T cells of the humanized CSPG4 chimeric antigen receptor engineered and optimized in the present disclosure released higher levels of cytokines during killing compared to the lower Th1-type cytokines (IFN-gamma, IL-2) released by the murine receptor, indicating that the humanization engineered and structurally-optimized CSPG4 chimeric antigen receptor have better killing effect on tumors with high CSPG4 expression than the original murine receptor.

### Example 8: Survival Experiments of Xenografted Mice

To determine the tumor killing effect of the T cells of the CSPG4 humanized chimeric antigen receptor engineered and optimized in the present disclosure in vivo, a mice brain glioma model was selected. Luciferase-labeled tumor cells were injected intracerebrally into nude mice, control (CD19-targeting chimeric antigen receptor) and CSPG4-targeting humanized chimeric antigen receptor T cells were injected intraventricularly after tumor formation, and tumor clearance by T cells and survival of treated mice were detected by imaging.

Method: the in vivo experiment of CSPG4 humanized chimeric antigen receptor—T cells targeting to brain gliomas were performed in nude mice at 6-8 weeks. GFP-FFLuc labeled tumor cell lines were stereotaxically implanted in the brain of nude mice, and 2-3 weeks later, once successful tumor implantation was confirmed in an in vivo imaging system, control or CSPG4-targeting humanized chimeric antigen receptor—T cells were injected intraventricularly. Tumor killing by T cells was observed by weekly imaging.

In vivo survival experiments, GFP-FFLuc-labeled LN-229 cells were stereotaxically injected into the brain of mice, 44 days later, the solvent PBS, control CD19-CAR T cells and CSPG4-targeting mouse (mCSPG4) and humanized (hCSPG4-HL, hCSPG4-LH) CAR-T cells were intraventricularly injected. The body weight of the mice was measured weekly thereafter and the time of death of the mice was recorded.

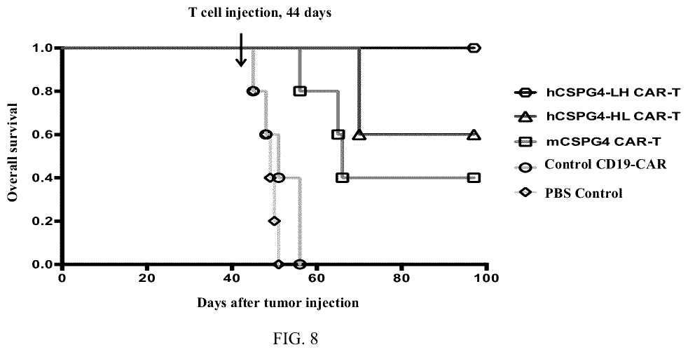

Results: as shown in FIG. 7, the T cells of the CSPG4 chimeric antigen receptors engineered and optimized in the present disclosure can clear tumors in most mice and control tumor recurrence within a certain period of time, as compared to the continued tumor growth of the control group. This result indicates that T cells of CSPG4-targeting humanized chimeric antigen receptors can kill tumors and control tumor recurrence in vivo.

Survival of LN-229 brain glioma mice undergoing control and T cell therapy of CSPG4-targeting chimeric antigen receptors was also detected. As shown in FIG. 8, compared with control PBS and CD19-CAR-T, murine CSPG4 CAR-T (mCSPG4) can prolong the survival time of mice to a certain extent, while the humanized CSPG4 CAR-T of the present disclosure (hCSPG4-HL and hCSPG4-LH) achieves better therapeutic effect than mouse CAR-T. In particular, the structurally optimized hCSPG4-LH CAR-T significantly prolongs the survival time of mice. This demonstrates that the humanized CSPG4 CAR-T of the present disclosure can exhibit better therapeutic effect on tumors than the original mouse CAR-T.

What is claimed is:

1. A CSPG4-targeting humanized chimeric antigen receptor (CAR) comprising: an anti-CSPG4 binding domain, a hinge region, a transmembrane domain and a signal transduction domain, the anti-CSPG4 binding domain comprises an anti-CSPG4 antibody or antigen binding part comprising a heavy chain CDR selected from amino acid sequences shown in SEQ ID NOS: 5-7 or any variant thereof, and/or a light chain CDR selected from amino acid sequences shown in SEQ ID NOS: 10-12 or any variant thereof, characterized in that the anti-CSPG4 binding domain is humanized.

2. The humanized chimeric antigen receptor (CAR) of claim 1, wherein the anti-CSPG4 binding domain is humanized means that, the variable region framework of the anti-CSPG4 binding domain is humanized.

3. The humanized chimeric antigen receptor (CAR) of claim 1, wherein the variable region framework of the anti-CSPG4 binding domain is humanized means that, the heavy chain variable region framework comprises a heavy chain FR selected from amino acid sequences shown in SEQ ID NOS: 16-19 or any variant thereof; and/or
the light chain variable region framework comprises a light chain FR selected from amino acid sequences shown in SEQ ID NOS: 20-23 or any variant thereof.

4. The humanized chimeric antigen receptor (CAR) of claim 1, wherein the variable region framework of the anti-CSPG4 binding domain is humanized means that, the heavy chain variable region framework comprises a heavy chain FR1 selected from the amino acid sequence shown in SEQ ID NO: 16 or any variant thereof, a heavy chain FR2 selected from the amino acid sequence shown in SEQ ID NO: 17 or any variant thereof, a heavy chain FR3 selected from the amino acid sequence shown in SEQ ID NO: 18 or any variant thereof, a heavy chain FR4 selected from the amino acid sequence shown in SEQ ID NO: 19 or any variant thereof; and/or
a light chain FR1 selected from the amino acid sequence shown in SEQ ID NO: 20 or any variant thereof, a light chain FR2 selected from the amino acid sequence shown in SEQ ID NO: 21 or any variant thereof, a light chain FR3 selected from the amino acid sequence shown in SEQ ID NO: 22 or any variant thereof, a light chain FR4 selected from the amino acid sequence shown in SEQ ID NO: 23 or any variant thereof.

5. The humanized chimeric antigen receptor (CAR) of claim 1, wherein the anti-CSPG4 binding domain comprises an anti-CSPG4 antibody or antigen binding part comprising a heavy chain CDR1 selected from the amino acid sequence shown in SEQ ID NO: 5 or any variant thereof, a heavy chain CDR2 selected from the amino acid sequence shown in SEQ ID NO: 6 or any variant thereof, a heavy chain CDR3 selected from the amino acid sequence shown in SEQ ID NO: 7 or any variant thereof; and/or
a light chain CDR1 selected from the amino acid sequence shown in SEQ ID NO: 10 or any variant thereof, a light chain CDR2 selected from the amino acid sequence shown in SEQ ID NO: 11 or any variant thereof, a light chain CDR3 selected from the amino acid sequence shown in SEQ ID NO: 12 or any variant thereof.

6. The humanized chimeric antigen receptor (CAR) of claim 1, the anti-CSPG4 binding domain comprising an anti-CSPG4 antibody or antigen binding part comprising a heavy chain variable region sequence selected from the amino acid sequence shown in SEQ ID NO: 4 or any variant thereof; and/or
a light chain variable region sequence selected from the amino acid sequence shown in SEQ ID NO: 9 or any variant thereof.

7. The humanized chimeric antigen receptor (CAR) of claim 1, wherein the anti-CSPG4 binding domain comprises an anti-CSPG4 single-chain antibody (scFv), preferably the amino acid sequence of the anti-CSPG4 single-chain antibody is selected from amino acid sequences shown in SEQ ID NOS: 2, 14 or any variant thereof.

8. The humanized chimeric antigen receptor (CAR) of claim 7, wherein the heavy and light chains of the anti-CSPG4 single-chain antibody are operably linked by a linker, preferably the linker comprises an amino acid sequence selected from the amino acid sequence shown in SEQ ID NO: 15 or any variant thereof.

9. The humanized chimeric antigen receptor (CAR) of claim 1, wherein the transmembrane domain is selected from one or more of the α, β, ζ chain of TCR, CD3γ, CD3δ, CD3ε, CD3ζ, CD3γ, CD3δ, CD4, CD5, CD8α, CD8β, CD9, CD16, CD22, CD27, CD28, CD33, CD37, CD45, CD64, CD80, CD86, CD134, 4-1BB, CD152, CD154, PD-1, NKp44, NKp46, and NKG2D transmembrane domains; preferably, the transmembrane domain is selected from one or more of the CD8α, CD8β, CD4, CD45, PD-1, CD154, and CD28 transmembrane domains; preferably, the transmembrane domain is selected from one or more of the CD8α, CD28 transmembrane domains; more preferably, the amino acid sequence of the transmembrane domain is selected from the amino acid sequence of SEQ ID NO: 27 or any variant thereof;
the hinge region is selected from one or more of a CD8 extracellular hinge region of CD8, an IgG1 Fc CH2CH3 hinge region, an IgD hinge region, a CD28 extracellular hinge region, an IgG4 Fc CH2CH3 hinge region and a CD4 extracellular hinge region; preferably, the hinge region is the CD8α hinge region; more preferably, the amino acid sequence of the hinge region is selected from the amino acid sequence of SEQ ID NO: 25 or any variant thereof.

10. The humanized chimeric antigen receptor (CAR) of claim 1, wherein the signal transduction domain is selected from one or more of TCRξ, FcRγ, FcRβ, CD3γ, CD3δ, CD3ε, CD5, CD22, CD79a, CD79b, CD278(ICOS), CD66d, DAP10, DAP12, and CD3ζ intracellular signal regions; preferably, the signal transduction domain is selected from the CD3ζ intracellular signal region; more preferably, the amino acid sequence of the signal transduction domain is selected from the amino acid sequence of SEQ ID NO: 33 or any variant thereof;
preferably, the signal transduction domain further comprises one or more costimulatory domains;
preferably, the costimulatory domains are selected from one or more of CARD11, CD2, CD7, CD27, CD28, CD30, CD40, CD54, CD83, OX40, 4-1BB, CD134, CD150, CD152, CD223, CD270, PD-L2, PD-L1, CD278, DAP10, DAP12, LAT, NKD2C, SLP76, TRIM, FcεRIγ, MyD88, 4-1BBL, and 2B4 intracellular signal regions; preferably, the costimulatory domains are selected from the 4-1BB, CD134, CD28, and OX40 intracellular signal regions; preferably, the costimulatory domains are selected from one or more of the 4-1BB and CD28 intracellular signal domain; more preferably, the amino acid sequences of the costimulatory domains are selected from the amino acid sequences of SEQ ID NOS: 29, 31 or any variant thereof.

11. An isolated nucleic acid molecule comprising the polynucleotide sequence encoding the humanized chimeric antigen receptor (CAR) of claim 1; preferably, the nucleotide sequence of the nucleic acid molecule is selected from the nucleotide sequences of SEQ ID NOS: 1, 13 or any variant thereof.

12. A nucleic acid construct comprising the nucleic acid molecule of claim 11; preferably, the nucleic acid construct is a viral vector; more preferably, the viral vector is one or more of a retroviral vector, a lentiviral vector, an adenoviral vector, and an adeno-associated viral vector.

13. A virus comprising the nucleic acid molecule comprising the polynucleotide sequence encoding the humanized chimeric antigen receptor (CAR) of claim 1 or the nucleic acid construct comprising the nucleic acid molecule; preferably, the virus is one or more of a retrovirus, a lentivirus, an adenovirus, and an adeno-associated virus.

14. Uses of the humanized chimeric antigen receptor (CAR) of claim 1, the nucleic acid molecule comprising the polynucleotide sequence encoding the humanized chimeric antigen receptor (CAR), the nucleic acid construct comprising the nucleic acid molecule, and the virus comprising the nucleic acid molecule or the nucleic acid construct, in the preparation of genetically modified immune cells targeting tumor cells expressing CSPG4.

15. An isolated host cell expressing the humanized chimeric antigen receptor (CAR) of claim 1, or expressing the isolated nucleic acid molecule comprising the polynucleotide sequence encoding the humanized chimeric antigen receptor (CAR), or expressing the nucleic acid construct comprising the nucleic acid molecule, or expressing the virus comprising the nucleic acid molecule or the nucleic acid construct; preferably, the host cell is a mammalian cell; more preferably, the host cell is one or more of a T cell, an NK cell, an γδT cell, an NKT cell, a macrophage or cell line, a PG13 cell line, a 293 cell line and cell lines derived therefrom; more preferably, the host cell is a T cell; most preferably, the host cell is a primarily cultured T cell.

16. The host cell of claim 15, the host cell further expresses other effector molecules, preferably the other effector molecules comprise one or more of cytokines, chemokines, another chimeric antigen receptor (CAR), chemokine receptors, siRNA/shRNAs or sgRNAs knocking down or knocking out PD-1 expression or proteins blocking PD-L1, TCRs, and safety switches;
preferably, the cytokine is selected from one or more of TNF-α, TNF-β, VEGF, TPO, NGF-β, PDGF, TGF-α, TGF-β, IGF-I, IGF-II, EPO, M-CSF, IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25 [symbol] LIF [symbol] FLT-3, interferon, angiostatin, thrombospondin, and endostatin;
preferably, the chemokine is selected from one or more of CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL3L3, CCL4, CCL4L1, CCL5, CCL6, CCL7, CCL8, CCL9, CX3CL1, CXCL1, CXCL10, CXCL11, CXCL12, CXCL13, CXCL14, CXCL15, CXCL16, CXCL17, CXCL2, CXCL3, CXCL4, CXCL5, CXCL6, CXCL7, CXCL9, CXCL8, XCL1, XCL2, FAM19A1, FAM19A2, FAM19A3, FAM19A4, and FAM19A5;
preferably, the chemokine receptor is selected from one or more of CCR1, CCR2, CCR3, CCR4, CCR5, CCR6, CCR7, CCR8, CCRL1, CXCR3, CXCR4, CXCR5, CXCR6, CXCR7, CXCR1, and CXCR2;
preferably, the safety switch is selected from one or more of HSVTK, VZVTK, iCaspase-9, iCaspase-1, iCaspase-8, truncated EGFR, and RQR8.

17. A pharmaceutical composition comprising the humanized chimeric antigen receptor (CAR) of claim 1, the nucleic acid molecule comprising the polynucleotide sequence encoding the humanized chimeric antigen receptor (CAR), the nucleic acid construct comprising the nucleic acid molecule, the virus comprising the nucleic acid molecule or the nucleic acid construct and/or the host cell expressing the humanized chimeric antigen receptor (CAR) of claim 1, and a pharmaceutically acceptable carrier.

18. Applications of the humanized chimeric antigen receptor (CAR) of claim 1, the nucleic acid molecule comprising the polynucleotide sequence encoding the humanized chimeric antigen receptor (CAR), the nucleic acid construct comprising the nucleic acid molecule, the virus comprising the nucleic acid molecule or the nucleic acid construct, the host cell expressing the humanized chimeric antigen receptor (CAR) of claim 1 and/or the pharmaceutical composition comprising the humanized chimeric antigen receptor (CAR) of claim 1 in the preparation of a medicament; preferably, the medicament is used in diagnosing, treating or preventing cancer-related diseases;
preferably, the medicament is used in diagnosing, treating or preventing tumors expressing CSPG4; more preferably, the medicament is used in diagnosing, treating or preventing one or more selected from brain cancer, breast cancer, head and neck cancer, melanoma, and mesothelioma; more preferably, the brain cancer is selected from one or more of brain glioblastoma, astrocytoma, meningioma, oligodendroglioma, and glioma, the breast cancer is triple negative breast cancer, the head and neck cancer is head and neck squamous cell carcinoma.
